SEQUENCE LISTING (27 sequences)

SEQ ID NO: 1 - protein, 15 aa, Artificial
  f88-4/15-mer phage-display library
EGVLGAALSAFSFDS

SEQ ID NO: 2 - protein, 15 aa, Artificial
  f88-4/15-mer peptide phage-display library
QAQPCPNQPDGSVYA

SEQ ID NO: 3 - protein, 15 aa, Artificial
  f88-4/15-mer peptide phage-display library
STALPLWSNYSYDSA

SEQ ID NO: 4 - protein, 15 aa, Artificial
  f88-4/15-mer peptide phage-display library
RTFGAKPPNIPFPRR

SEQ ID NO: 5 - protein, 15 aa, Artificial
  f88-4/15-mer peptide phage-display library
KAIAFQSSPGSPVLL

SEQ ID NO: 6 - protein, 15 aa, Artificial
  f88-4/15-mer peptide phage-display library
TCNLSDYTLPRARVL

SEQ ID NO: 7 - protein, 15 aa, Artificial
  f88-4/15-mer peptide phage-display library
NFLCPSPYPKVQPGL

SEQ ID NO: 8 - protein, 15 aa, Artificial
  f88-4/15-mer peptide phage-display library
VPESIHKASLVCYRF

SEQ ID NO: 9 - protein, 15 aa, Artificial
  f88-4/15-mer peptide phage-display library
SRPIARFYTWSQNTT

SEQ ID NO: 10 - protein, 15 aa, Artificial
  f88-4/15-mer peptide phage-display library
FSPFRISELVYTLHP

SEQ ID NO: 11 - protein, 15 aa, Artificial
  f88-4/15-mer peptide phage-display library
LPFNLAKPELYIFVQ

SEQ ID NO: 12 - protein, 15 aa, Artificial
  f88-4/15-mer peptide phage-display library
GRFLIRVTSSPLGPD

SEQ ID NO: 13 - protein, 15 aa, Artificial
  f88-4/15-mer peptide phage-display library
TGSGLYLHQMVYLYQ

SEQ ID NO: 14 - protein, 15 aa, Artificial
  f88-4/15-mer peptide phage-display library
FLIDSPLASIGPTSM

SEQ ID NO: 15 - protein, 15 aa, Artificial
  f88-4/15-mer peptide phage-display library
FMIDSPLASIGPTSM

SEQ ID NO: 16 - protein, 15 aa, Artificial
  f88-4/15-mer peptide phage-display library
FLSDPPAPPTSPGVV

SEQ ID NO: 17 - protein, 15 aa, Artificial
  f88-4/15-mer peptide phage-display library
VLAWFSPLTLESSRL

SEQ ID NO: 18 - protein, 15 aa, Artificial
  f88-4/15-mer peptide phage-display library
VIDLSGTRKSSSGTM

SEQ ID NO: 19 - protein, 15 aa, Artificial
  f88-4/15-mer peptide phage-display library
VRKTTSHPPSYALLH

SEQ ID NO: 20 - protein, 15 aa, Artificial
  f88-4/15-mer peptide phage-display library
TPPYRAALATPVLLL

SEQ ID NO: 21 - protein, 15 aa, Artificial
  f88-4/15-mer peptide phage-display library
LSAPSPMFLPPVNPH

SEQ ID NO: 22 - protein, 15 aa, Artificial
  f88-4/15-mer peptide phage-display library
HRPVKTPANAPTTMM

SEQ ID NO: 23 - protein, 15 aa, Artificial
  f88-4/15-mer peptide phage-display library
CFNDPLDIVPPMLLL

SEQ ID NO: 24 - protein, 15 aa, Artificial
  f88-4/15-mer peptide phage-display library
AHAPCSLFFPLSLRP

SEQ ID NO: 25 - protein, 15 aa, Artificial
  f88-4/15-mer peptide phage-display library
KALYALHVPSMQVFA

SEQ ID NO: 26 - protein, 15 aa, Artificial
  f88-4/15-mer peptide phage-display library
RPSRWPWQEPLPISI

SEQ ID NO: 27 - protein, 15 aa, Artificial
  f88-4/15-mer peptide phage-display library
KVQIIPKDTLAPLPP

(19) United States
(12) Patent Application Publication
(10) Pub. No.: US 2010/0190719 A1
(43) Pub. Date: Jul. 29, 2010

(54) PEPTIDES AS ANTIMICROBIAL AGENTS TOWARDS CAMPYLOBACTER SPP

(75) Inventors: Sharon Lillian Bishop-Hurley, Queensland (AU); Christopher Simon McSweeney, Queensland (AU)

Correspondence Address: GRAYBEAL JACKSON LLP, 400 - 108TH AVENUE NE, SUITE 700, BELLEVUE, WA, 98004, US

(73) Assignee: Australian Poultry CRC Pty Ltd., Armidale, New South Wales (AU)

(21) Appl. No.: 12/452,247

(22) PCT Filed: Jun. 18, 2008
(86) PCT No.: PCT/AU2008/000878

§ 371 Date: Mar. 22, 2010

(30) Foreign Application Priority Data
Jun. 22, 2007 (AU) 2007903374

(51) Int. Cl. A61K 38/10; C07K 7/08; G01N 33/53; C07K 14/00; C12N 7/00; C07H 21/04; A61P 31/00

(52) U.S. Cl. 514/14; 530/326; 435/7.1; 530/350; 435/235.1; 536/23.1

(57) ABSTRACT

The present invention relates to peptides that exhibit antimicrobial properties towards Campylobacter spp, compositions comprising such peptides, the use of such peptides for inhibiting growth of or killing Campylobacter spp. and for the diagnosis, prevention and treatment of Campylobacter infection.

### FIELD OF THE INVENTION

The present invention relates to peptides that exhibit antimicrobial properties towards Campylobacter spp, compositions comprising such peptides, the use of such peptides for inhibiting growth of or killing Campylobacter spp. and for the diagnosis, prevention and treatment of Campylobacter infection.

### BACKGROUND OF THE INVENTION

Campylobacter jejuni is a leading cause of sporadic food-borne illness in humans (Altekruse et al. 1999; Wren et al. 2001). The majority of diagnosed Campylobacter infections are due to C. jejuni (95%) with Campylobacter coli being a less frequent cause of disease. For most people, infection usually results in a moderate diarrhoeal disease; however, severe and prolonged illness is likely to occur in people with weakened, under-developed or suppressed immune systems (e.g. children, elderly and people with chronic illnesses). These cases often require therapeutic intervention; however, over the last decade C. jejuni has become increasingly resistant to antibiotics such as tetracycline, nalidixic acid and fluorquinolones (Prats et al. 2000; Ge et al. 2003; Gupta et al. 2004), making infections harder to treat. In approximately 0.1% of cases, C. jejuni infection can lead to chronic illnesses such as Guillain-Barré Syndrome (GBS), Miller-Fisher Syndrome or reactive arthritis.

Chicken meat is a major source of infection for humans (Wallace et al. 1998; Altekruse et al. 1999), with the handling and consumption of raw and under-cooked poultry products being a major risk factor for campylobacteriosis in humans. Retail chicken meat is often surface contaminated with thermophilic Campylobacter spp., with levels of contamination reported to be between 10³-10⁷ CFU/carcass (Cason et al. 1997; Altekruse et al. 1999). In contrast, a lower prevalence of Campylobacter spp. have been isolated from other meats (e.g. beef, 0.5%; pork 1.5%) (Zhao et al. 2001).

Campylobacter resides as part of the normal gastrointestinal microflora species of livestock species such as poultry, with approximately 70-90% of poultry flocks becoming colonised by 4-7 weeks of age (Denis et al. 2001; Evans & Sayers 2000). Various studies carried out at abattoirs indicate that during processing, the principal spread of C. jejuni is from the gastrointestinal tract onto the surface of dressed carcasses (Rivoal et al. 1999; Berrang et al. 2000; Musgrove et al. 1997). Its subsequent removal from meat products is incredibly difficult due to the nature of its interaction with the skin (Northcutt et al. 2004).

Current strategies for preventing Campylobacter colonisation are proving ineffective. For example, Campylobacter is ubiquitously present in many farm animals, pets and birds, making infection in a commercial setting almost impossible to prevent through conventional biosecurity measures. Several other prevention strategies proposed to reduce colonisation in chickens do not offer much in practical significance. For example, probiotic treatment of chickens with lactic acid bacteria and competitive exclusion with beneficial microflora only reduces colonisation by 2-27% (Morishita et al. 1997; Stern et al. 2005). Conventional experimental vaccines are also hampered by the large serotype diversity of C. jejuni strains. Current antibiotics, designed for human medicine, generally have a broad-spectrum of activity and fall into a relatively small number of classes with respect to mechanisms of action. Consequently, chickens fed these antibiotics can be a reservoir for antibiotic-resistant bacteria in the gastrointestinal tract, which could be transmitted to humans via contaminated poultry in the retail store.

There remains a need for alternative antimicrobial compounds for reducing colonisation and growth of Campylobacter spp. and for diagnosis of infection.

### SUMMARY OF THE INVENTION

In one aspect, the present invention relates to an isolated peptide which binds to Campylobacter spp., wherein the peptide is selected from the group consisting of: a) a peptide comprising the amino acid sequence EGVLGAALSAFSFDS (SEQ ID NO: 1) or a fragment thereof which is capable of binding to Campylobacter spp; b) a peptide comprising the amino acid sequence QAQPCPNQPDGSVYA (SEQ ID NO: 2) or a fragment thereof which is capable of binding to Campylobacter spp; c) a peptide comprising the amino acid sequence STALPLWSNYSYDSA (SEQ ID NO: 3) or a fragment thereof which is capable of binding to Campylobacter spp; d) a peptide comprising the amino acid sequence RTFGAKPPNIPFPRR (SEQ ID NO: 4) or a fragment thereof which is capable of binding to Campylobacter spp; e) a peptide comprising the amino acid sequence KAIAFQSSPGSPVLL (SEQ ID NO: 5) or a fragment thereof which is capable of binding to Campylobacter spp; f) a peptide comprising the amino acid sequence TCNLSDYTLPRARVL (SEQ ID NO: 6) or a fragment thereof which is capable of binding to Campylobacter spp; g) a peptide comprising the amino acid sequence NFLCPSPYPKVQPGL (SEQ ID NO: 7) or a fragment thereof which is capable of binding to Campylobacter spp; h) a peptide comprising the amino acid sequence VPESIHKASLVCYRF (SEQ ID NO: 8) or a fragment thereof which is capable of binding to Campylobacter spp; i) a peptide comprising the amino acid sequence SRPIARFYTWSQNTT (SEQ ID NO: 9) or a fragment thereof which is capable of binding to Campylobacter spp; j) a peptide comprising the amino acid sequence FSPFRISELVYTLHP (SEQ ID NO: 10) or a fragment thereof which is capable of binding to Campylobacter spp; k) a peptide comprising the amino acid sequence LPFNLAKPELYIFVQ (SEQ ID NO: 11) or a fragment thereof which is capable of binding to Campylobacter spp; 1) a peptide comprising the amino acid sequence GRFLIRVTSSPLGPD (SEQ ID NO: 12) or a fragment thereof which is capable of binding to Campylobacter spp; m) a peptide comprising the amino acid sequence TGSGLYLHQMVYLYQ (SEQ ID NO: 13) or a fragment thereof which is capable of binding to Campylobacter spp; n) a peptide comprising the amino acid sequence FLIDSPLASIGPTSM (SEQ ID NO: 14) or a fragment thereof which is capable of binding to Campylobacter spp; o) a peptide comprising the amino acid sequence FMIDSPLASIGPTSM (SEQ ID NO: 15) or a fragment thereof which is capable of binding to Campylobacter spp; p) a peptide comprising the amino acid sequence FLSDPPAPPTSPGVV (SEQ ID NO: 16) or a fragment thereof which is capable of binding to Campylobacter spp; q) a peptide comprising the amino acid sequence VLAWFSPLTLESSRL (SEQ ID NO: 17) or a fragment thereof which is capable of binding to Campylobacter spp; r) a peptide comprising the amino acid sequence VIDLSGTRKSSSGTM (SEQ ID NO: 18) or a fragment thereof which is capable of binding to Campylobacter spp; s) a peptide comprising the amino acid sequence VRKTTSHPPSYALLH (SEQ ID NO: 19) or a fragment thereof which is capable of binding to Campylobacter spp; t) a peptide comprising the amino acid sequence TPPYRAALATPVLLL (SEQ ID NO: 20) or a fragment thereof which is capable of binding to Campylobacter spp; u) a peptide comprising the amino acid sequence LSAPSPMFLPPVNPH (SEQ ID NO: 21) or a fragment thereof which is capable of binding to Campylobacter spp; v) a peptide comprising the amino acid sequence HRPVKTPANAPTTMM (SEQ ID NO: 22) or a fragment thereof which is capable of binding to Campylobacter spp; w) a peptide comprising the amino acid sequence CFNDPLDIVPPMLLL (SEQ ID NO: 23) or a fragment thereof which is capable of binding to Campylobacter spp; x) a peptide comprising the amino acid sequence AHAPCSLFFPLSLRP (SEQ ID NO: 24) or a fragment thereof which is capable of binding to Campylobacter spp; y.) a peptide comprising the amino acid sequence KALYALHVPSMQVFA (SEQ ID NO: 25) or a fragment thereof which is capable of binding to Campylobacter spp; z) a peptide comprising the amino acid sequence RPSRWPWQEPLPISI (SEQ ID NO: 26) or a fragment thereof which is capable of binding to Campylobacter spp; and aa) a peptide comprising the amino acid sequence KVQIIPKDTLAPLPP (SEQ ID NO: 27) or a fragment thereof which is capable of binding to Campylobacter spp.

In one embodiment, the peptide binds specifically to Campylobacter spp.

In another aspect, the present invention relates to a method of inhibiting growth of or killing a Campylobacter species comprising contacting the species with one or more peptides in an amount sufficient to inhibit growth of or kill the species, wherein the one or more peptides are selected from the group consisting of: a) a peptide comprising the amino acid sequence EGVLGAALSAFSFDS (SEQ ID NO: 1) or a fragment thereof which is capable of inhibiting growth of or killing Campylobacter spp; b) a peptide comprising the amino acid sequence QAQPCPNQPDGSVYA (SEQ ID NO: 2) or a fragment thereof which is capable of inhibiting growth of or killing Campylobacter spp; c) a peptide comprising the amino acid sequence STALPLWSNYSYDSA (SEQ ID NO: 3) or a fragment thereof which is capable of inhibiting growth of or killing Campylobacter spp; d) a peptide comprising the amino acid sequence RTFGAKPPNIPFPRR (SEQ ID NO: 4) or a fragment thereof which is capable of inhibiting growth of or killing Campylobacter spp; e) a peptide comprising the amino acid sequence KAIAFQSSPGSPVLL (SEQ ID NO: 5) or a fragment thereof which is capable of inhibiting growth of or killing Campylobacter spp; f) a peptide comprising the amino acid sequence TCNLSDYTLPRARVL (SEQ ID NO: 6) or a fragment thereof which is capable of inhibiting growth of or killing Campylobacter spp; g) a peptide comprising the amino acid sequence NFLCPSPYPKVQPGL (SEQ ID NO: 7) or a fragment thereof which is capable of inhibiting growth of or killing Campylobacter spp; h) a peptide comprising the amino acid sequence VPESIHKASLVCYRF (SEQ ID NO: 8) or a fragment thereof which is capable of inhibiting growth of or killing Campylobacter spp; i) a peptide comprising the amino acid sequence SRPIARFYTWSQNTT (SEQ ID NO: 9) or a fragment thereof which is capable of inhibiting growth of or killing Campylobacter spp; j) a peptide comprising the amino acid sequence FSPFRISELVYTLHP (SEQ ID NO: 10) or a fragment thereof which is capable of inhibiting growth of or killing Campylobacter spp; k) a peptide comprising the amino acid sequence LPFNLAKPELYIFVQ (SEQ ID NO: 11) or a fragment thereof which is capable of inhibiting growth of or killing Campylobacter spp; 1) a peptide comprising the amino acid sequence GRFLIRVTSSPLGPD (SEQ ID NO: 12) or a fragment thereof which is capable of inhibiting growth of or killing Campylobacter spp; m) a peptide comprising the amino acid sequence TGSGLYLHQMVYLYQ (SEQ ID NO: 13) or a fragment thereof which is capable of inhibiting growth of or killing Campylobacter spp; n) a peptide comprising the amino acid sequence FLIDSPLASIGPTSM (SEQ ID NO: 14) or a fragment thereof which is capable of inhibiting growth of or killing Campylobacter spp; o) a peptide comprising the amino acid sequence FMIDSPLASIGPTSM (SEQ ID NO: 15) or a fragment thereof which is capable of inhibiting growth of or killing Campylobacter spp; p) a peptide comprising the amino acid sequence FLSDPPAPPTSPGVV (SEQ ID NO: 16) or a fragment thereof which is capable of inhibiting growth of or killing Campylobacter spp; q) a peptide comprising the amino acid sequence VLAWFSPLTLESSRL (SEQ ID NO: 17) or a fragment thereof which is capable of inhibiting growth of or killing Campylobacter spp; r) a peptide comprising the amino acid sequence VIDLSGTRKSSSGTM (SEQ ID NO: 18) or a fragment thereof which is capable of inhibiting growth of or killing Campylobacter spp; s) a peptide comprising the amino acid sequence VRKTTSHPPSYALLH (SEQ ID NO: 19) or a fragment thereof which is capable of inhibiting growth of or killing Campylobacter spp; t) a peptide comprising the amino acid sequence TPPYRAALATPVLLL (SEQ ID NO: 20) or a fragment thereof which is capable of inhibiting growth of or killing Campylobacter spp; u) a peptide comprising the amino acid sequence LSAPSPMFLPPVNPH (SEQ ID NO: 21) or a fragment thereof which is capable of inhibiting growth of or killing Campylobacter spp; v) a peptide comprising the amino acid sequence HRPVKTPANAPTTMM (SEQ ID NO: 22) or a fragment thereof which is capable of inhibiting growth of or killing Campylobacter spp; w) a peptide comprising the amino acid sequence CFNDPLDIVPPMLLL (SEQ ID NO: 23) or a fragment thereof which is capable of inhibiting growth of or killing Campylobacter spp; x) a peptide comprising the amino acid sequence AHAPCSLFFPLSLRP (SEQ ID NO: 24) or a fragment thereof which is capable of inhibiting growth of or killing Campylobacter spp; y) a peptide comprising the amino acid sequence KALYALHVPSMQVFA (SEQ ID NO: 25) or a fragment thereof which is capable of inhibiting growth of or killing Campylobacter spp; z) a peptide comprising the amino acid sequence RPSRWPWQEPLPISI (SEQ ID NO: 26) or a fragment thereof which is capable of inhibiting growth of or killing Campylobacter spp; and aa) a peptide comprising the amino acid sequence KVQIIPKDTLAPLPP (SEQ ID NO: 27) or a fragment thereof which is capable of inhibiting growth of or killing Campylobacter spp.

In one embodiment, the one or more peptides bind to the Campylobacter species to inhibit growth of the Campylobacter species. The peptide may bind specifically to the Campylobacter spp to inhibit growth of the Campylobacter species.

In one embodiment, the one or more peptides bind to the Campylobacter species to kill the Campylobacter species. The peptide may bind specifically to the Campylobacter spp to kill the Campylobacter species.

The Campylobacter spp. may be any species of Campylobacter. In one embodiment, the Campylobacter spp. is Campylobacter jejuni. In another embodiment, the Campylobacter spp. is Campylobacter coli.

In one embodiment, the one or more peptides is a peptide comprising the amino acid sequence EGVLGAALSAFSFDS (SEQ ID NO: 1), or a fragment thereof which is capable of inhibiting growth of or killing Campylobacter spp. In one embodiment, the one or more peptides is a peptide comprising the amino acid sequence QAQPCPNQPDGSVYA (SEQ ID NO: 2) or a fragment thereof which is capable of inhibiting growth of or killing Campylobacter spp. In one embodiment, the one or more peptides is a peptide comprising the amino acid sequence STALPLWSNYSYDSA (SEQ ID NO: 3) or a fragment thereof which is capable of inhibiting growth of or killing Campylobacter spp. In one embodiment, the one or more peptides is a peptide comprising the amino acid sequence RTFGAKPPNIPFPRR (SEQ ID NO: 4) or a fragment thereof which is capable of inhibiting growth of or killing Campylobacter spp. In one embodiment, the one or more peptides is a peptide comprising the amino acid sequence KAIAFQSSPGSPVLL (SEQ ID NO: 5) or a fragment thereof which is capable of inhibiting growth of or killing Campylobacter spp. In one embodiment, the one or more peptides is a peptide comprising the amino acid sequence TCNLSDYTLPRARVL (SEQ ID NO: 6) or a fragment thereof which is capable of inhibiting growth of or killing Campylobacter spp. In one embodiment, the one or more peptides is a peptide comprising the amino acid sequence NFLCPSPYPKVQPGL (SEQ ID NO: 7) or a fragment thereof which is capable of inhibiting growth of or killing Campylobacter spp. In one embodiment, the one or more peptides is a peptide comprising the amino acid sequence VPESIHKASLVCYRF (SEQ ID NO: 8) or a fragment thereof which is capable of inhibiting growth of or killing Campylobacter spp. In one embodiment, the one or more peptides is a peptide comprising the amino acid sequence SRPIARFYTWSQNTT (SEQ ID NO: 9) or a fragment thereof which is capable of inhibiting growth of or killing Campylobacter spp. In one embodiment, the one or more peptides is a peptide comprising the amino acid sequence FSPFRISELVYTLHP (SEQ ID NO: 10) or a fragment thereof which is capable of inhibiting growth of or killing Campylobacter spp. In one embodiment, the one or more peptides is a peptide comprising the amino acid sequence LPFNLAKPELYIFVQ (SEQ ID NO: 11) or a fragment thereof which is capable of inhibiting growth of or killing Campylobacter spp. In one embodiment, the one or more peptides is a peptide comprising the amino acid sequence GRFLIRVTSSPLGPD (SEQ ID NO: 12) or a fragment thereof which is capable of inhibiting growth of or killing Campylobacter spp.

In one embodiment, the one or more peptides is a peptide comprising the amino acid sequence TGSGLYLHQMVYLYQ (SEQ ID NO: 13) or a fragment thereof which is capable of inhibiting growth of or killing Campylobacter spp. In one embodiment, the one or more peptides is a peptide comprising the amino acid sequence FLIDSPLASIGPTSM (SEQ ID NO: 14) or a fragment thereof which is capable of inhibiting growth of or killing Campylobacter spp. In one embodiment, the one or more peptides is a peptide comprising the amino acid sequence FMIDSPLASIGPTSM (SEQ ID NO: 15) or a fragment thereof which is capable of inhibiting growth of or killing Campylobacter spp. In one embodiment, the one or more peptides is a peptide comprising the amino acid sequence FLSDPPAPPTSPGVV (SEQ ID NO: 16), or a fragment thereof which is capable of inhibiting growth of or killing Campylobacter spp. In one embodiment, the one or more peptides is a peptide comprising the amino acid sequence VLAWFSPLTLESSRL (SEQ ID NO: 17) or a fragment thereof which is capable of inhibiting growth of or killing Campylobacter spp. In one embodiment, the one or more peptides is a peptide comprising the amino acid sequence VIDLSGTRKSSSGTM (SEQ ID NO: 18) or a fragment thereof which is capable of inhibiting growth of or killing Campylobacter spp. In one embodiment, the one or more peptides is a peptide comprising the amino acid sequence VRKTTSHPPSYALLH (SEQ ID NO: 19) or a fragment thereof which is capable of inhibiting growth of or killing Campylobacter spp. In one embodiment, the one or more peptides is a peptide comprising the amino acid sequence TPPYRAALATPVLLL (SEQ ID NO: 20) or a fragment thereof which is capable of inhibiting growth of or killing Campylobacter spp. In one embodiment, the one or more peptides is a peptide comprising the amino acid sequence LSAPSPMFLPPVNPH (SEQ ID NO: 21) or a fragment thereof which is capable of inhibiting growth of or killing Campylobacter spp. In one embodiment, the one or more peptides is a peptide comprising the amino acid sequence HRPVKTPANAPTTMM (SEQ ID NO: 22) or a fragment thereof which is capable of inhibiting growth of or killing Campylobacter spp. In one embodiment, the one or more peptides is a peptide comprising the amino acid sequence CFNDPLDIVPPMLLL (SEQ ID NO: 23) or a fragment thereof which is capable of inhibiting growth of or killing Campylobacter spp. In one embodiment, the one or more peptides is a peptide comprising the amino acid sequence AHAPCSLFFPLSLRP (SEQ ID NO: 24) or a fragment thereof which is capable of inhibiting growth of or killing Campylobacter spp. In one embodiment, the one or more peptides is a peptide comprising the amino acid sequence KALYALHVPSMQVFA (SEQ ID NO: 25) or a fragment thereof which is capable of inhibiting growth of or killing Campylobacter spp. In one embodiment, the one or more peptides is a peptide comprising the amino acid sequence RPSRWPWQEPLPISI (SEQ ID NO: 26) or a fragment thereof which is capable of inhibiting growth of or killing Campylobacter spp. In one embodiment, the one or more peptides is a peptide comprising the amino acid sequence KVQIIPKDTLAPLPP (SEQ ID NO: 27) or a fragment thereof which is capable of inhibiting growth of or killing Campylobacter spp.

In one embodiment, the method comprises inhibiting growth of or killing Campylobacter spp. in a solution. The solution may be any solution which is capable of becoming contaminated with Campylobacter spp. In one embodiment the solution is a solution produced in chicken processing or a solution associated with chicken production such as drinking water, water in air-conditioning units and so on.

In another embodiment, the method comprises inhibiting growth of or killing Campylobacter spp. on a surface. The surface may be any surface which is capable of becoming contaminated with Campylobacter spp. The surface may be a surface of agricultural equipment or equipment for use on poultry farms such as crates for transporting chickens, cages, food dishes and equipment for use in slaughter houses e.g equipment used for slaughtering chickens; on poultry meat and on the packaging. For example, the surface may be a surface of a piece of equipment that comes into contact with poultry or meat products. The surface may be a surface of an animal. In one embodiment, the surface is a surface of animal meat. For example, chicken meat.

In another aspect, the present invention relates to a method of preventing a Campylobacter spp. infection in a subject comprising administering to the subject one or more peptides in an amount sufficient to prevent infection in the subject, wherein the one or more peptides are selected from the group consisting of: a) a peptide comprising the amino acid sequence EGVLGAALSAFSFDS (SEQ ID NO: 1) or a fragment thereof which is capable of preventing Campylobacter spp infection; b) a peptide comprising the amino acid sequence QAQPCPNQPDGSVYA (SEQ ID NO: 2) or a fragment thereof which is capable of preventing Campylobacter spp infection; c) a peptide comprising the amino acid sequence STALPLWSNYSYDSA (SEQ ID NO: 3) or a fragment thereof which is capable of preventing Campylobacter spp infection; d) a peptide comprising the amino acid sequence RTFGAKPPNIPFPRR (SEQ ID NO: 4) or a fragment thereof which is capable of preventing Campylobacter spp infection; e) a peptide comprising the amino acid sequence KAIAFQSSPGSPVLL (SEQ ID NO: 5) or a fragment thereof which is capable of preventing Campylobacter spp infection; f) a peptide comprising the amino acid sequence TCNLSDYTLPRARVL (SEQ ID NO: 6) or a fragment thereof which is capable of preventing Campylobacter spp infection; g) a peptide comprising the amino acid sequence NFLCPSPYPKVQPGL (SEQ ID NO: 7) or a fragment thereof which is capable of preventing Campylobacter spp infection; h) a peptide comprising the amino acid sequence VPESIHKASLVCYRF (SEQ ID NO: 8) or a fragment thereof which is capable of preventing Campylobacter spp infection; i) a peptide comprising the amino acid sequence SRPIARFYTWSQNTT (SEQ ID NO: 9) or a fragment thereof which is capable of preventing Campylobacter spp infection; j) a peptide comprising the amino acid sequence FSPFRISELVYTLHP (SEQ ID NO: 10) or a fragment thereof which is capable preventing Campylobacter spp infection; k) a peptide comprising the amino acid sequence LPFNLAKPELYIFVQ (SEQ ID NO: 11) or a fragment thereof which is capable of preventing Campylobacter spp infection; 1) a peptide comprising the amino acid sequence GRFLIRVTSSPLGPD (SEQ ID NO: 12) or a fragment thereof which is capable of preventing Campylobacter spp infection; m) a peptide comprising the amino acid sequence TGSGLYLHQMVYLYQ (SEQ ID NO: 13) or a fragment thereof which is capable of preventing Campylobacter spp infection; n) a peptide comprising the amino acid sequence FLIDSPLASIGPTSM (SEQ ID NO: 14) or a fragment thereof which is capable of preventing Campylobacter spp infection; o) a peptide comprising the amino acid sequence FMIDSPLASIGPTSM (SEQ ID NO: 15) or a fragment thereof which is capable of preventing Campylobacter spp infection; p) a peptide comprising the amino acid sequence FLSDPPAPPTSPGVV (SEQ ID NO: 16) or a fragment thereof which is capable of preventing Campylobacter spp infection; q) a peptide comprising the amino acid sequence VLAWFSPLTLESSRL (SEQ ID NO: 17) or a fragment thereof which is capable of preventing Campylobacter spp infection; r) a peptide comprising the amino acid sequence VIDLSGTRKSSSGTM (SEQ ID NO: 18) or a fragment thereof which is capable of preventing Campylobacter spp infection; s) a peptide comprising the amino acid sequence VRKTTSHPPSYALLH (SEQ ID NO: 19) or a fragment thereof which is capable of preventing Campylobacter spp infection; t) a peptide comprising the amino acid sequence TPPYRAALATPVLLL (SEQ ID NO: 20) or a fragment thereof which is capable of preventing Campylobacter spp infection; u) a peptide comprising the amino acid sequence LSAPSPMFLPPVNPH (SEQ ID NO: 21) or a fragment thereof which is capable of preventing Campylobacter spp infection; v) a peptide comprising the amino acid sequence HRPVKTPANAPTTMM (SEQ ID NO: 22) or a fragment thereof which is capable of preventing Campylobacter spp infection; w) a peptide comprising the amino acid sequence CFNDPLDIVPPMLLL (SEQ ID NO: 23) or a fragment thereof which is capable of preventing Campylobacter spp infection; x) a peptide comprising the amino acid sequence AHAPCSLFFPLSLRP (SEQ ID NO: 24) or a fragment thereof which is capable of preventing Campylobacter spp infection; y) a peptide comprising the amino acid sequence KALYALHVPSMQVFA (SEQ ID NO: 25) or a fragment thereof which is capable of preventing Campylobacter spp infection; z) a peptide comprising the amino acid sequence RPSRWPWQEPLPISI (SEQ ID NO: 26) or a fragment thereof which is capable of preventing Campylobacter spp infection; and aa) a peptide comprising the amino acid sequence KVQIIPKDTLAPLPP (SEQ ID NO: 27) or a fragment thereof which is capable of preventing Campylobacter spp infection.

In another aspect, the present invention relates to a method of treating a Campylobacter spp. infection in a subject comprising administering to the subject one or more peptides in an amount sufficient to treat the infection in the subject, wherein the one or more peptides are selected from the group consisting of: a) a peptide comprising the amino acid sequence EGVLGAALSAFSFDS (SEQ ID NO: 1) or a fragment thereof which is capable of treating Campylobacter spp infection; b) a peptide comprising the amino acid sequence QAQPCPNQPDGSVYA (SEQ ID NO: 2) or a fragment thereof which is capable of treating Campylobacter spp infection; c) a peptide comprising the amino acid sequence STALPLWSNYSYDSA (SEQ ID NO: 3) or a fragment thereof which is capable of treating Campylobacter spp infection; d) a peptide comprising the amino acid sequence RTFGAKPPNIPFPRR (SEQ ID NO: 4) or a fragment thereof which is capable of treating Campylobacter spp infection; e) a peptide comprising the amino acid sequence KAIAFQSSPGSPVLL (SEQ ID NO: 5) or a fragment thereof which is capable of treating Campylobacter spp infection; f) a peptide comprising the amino acid sequence TCNLSDYTLPRARVL (SEQ ID NO: 6) or a fragment thereof which is capable of treating Campylobacter spp infection; g) a peptide comprising the amino acid sequence NFLCPSPYPKVQPGL (SEQ ID NO: 7) or a fragment thereof which is capable of treating Campylobacter spp infection; h) a peptide comprising the amino acid sequence VPESIHKASLVCYRF (SEQ ID NO: 8) or a fragment thereof which is capable of treating Campylobacter spp infection; i) a peptide comprising the amino acid sequence SRPIARFYTWSQNTT (SEQ ID NO: 9) or a fragment thereof which is capable of treating Campylobacter spp infection; j) a peptide comprising the amino acid sequence FSPFRISELVYTLHP (SEQ ID NO: 10) or a fragment thereof which is capable treating Campylobacter spp infection; k) a peptide comprising the amino acid sequence LPFNLAKPELYIFVQ (SEQ ID NO: 11) or a fragment thereof which is capable of treating Campylobacter spp infection; 1) a peptide comprising the amino acid sequence GRFLIRVTSSPLGPD (SEQ ID NO: 12) or a fragment thereof which is capable of treating Campylobacter spp infection; m) a peptide comprising the amino acid sequence TGSGLYLHQMVYLYQ (SEQ ID NO: 13) or a fragment thereof which is capable of treating Campylobacter spp infection; n) a peptide comprising the amino acid sequence FLIDSPLASIGPTSM (SEQ ID NO: 14) or a fragment thereof which is capable of treating Campylobacter spp infection, o) a peptide comprising the amino acid sequence FMIDSPLASIGPTSM (SEQ ID NO: 15) or a fragment thereof which is capable of treating Campylobacter spp infection; p) a peptide comprising the amino acid sequence FLSDPPAPPTSPGVV (SEQ ID NO: 16) or a fragment thereof which is capable of treating Campylobacter spp infection; q) a peptide comprising the amino acid sequence VLAWFSPLTLESSRL (SEQ ID NO: 17) or a fragment thereof. which is capable of treating Campylobacter spp infection; r) a peptide comprising the amino acid sequence VIDLSGTRKSSSGTM (SEQ ID NO: 18) or a fragment thereof which is capable of treating Campylobacter spp infection; s) a peptide comprising the amino acid sequence VRKTTSHPPSYALLH (SEQ ID NO: 19) or a fragment thereof which is capable of treating Campylobacter spp infection; t) a peptide comprising the amino acid sequence TPPYRAALATPVLLL (SEQ ID NO: 20) or a fragment thereof which is capable of treating Campylobacter spp infection; u) a peptide comprising the amino acid sequence LSAPSPMFLPPVNPH (SEQ ID NO: 21) or a fragment thereof which is capable of treating Campylobacter spp infection; v) a peptide comprising the amino acid sequence HRPVKTPANAPTTMM (SEQ ID NO: 22) or a fragment thereof which is capable of treating Campylobacter spp infection; w) a peptide comprising the amino acid sequence CFNDPLDIVPPMLLL (SEQ ID NO: 23) or a fragment thereof which is capable of treating Campylobacter spp infection; x) a peptide comprising the amino acid sequence AHAPCSLFFPLSLRP (SEQ ID NO: 24) or a fragment thereof which is capable of treating Campylobacter spp infection; y) a peptide comprising the amino acid sequence KALYALHVPSMQVFA (SEQ ID NO: 25) or a fragment thereof which is capable of treating Campylobacter spp infection; z) a peptide comprising the amino acid sequence RPSRWPWQEPLPISI (SEQ ID NO: 26) or a fragment thereof which is capable of treating Campylobacter spp infection; and aa) a peptide comprising the amino acid sequence KVQIIPKDTLAPLPP (SEQ ID NO: 27) or a fragment thereof which is capable of treating Campylobacter spp infection.

In another aspect, the present invention relates to use of one or more peptides selected from the group consisting of:

a) a peptide comprising the amino acid sequence EGVLGAALSAFSFDS (SEQ ID NO: 1) or a fragment thereof which is capable of treating Campylobacter spp infection;
b) a peptide comprising the amino acid sequence QAQPCPNQPDGSVYA (SEQ ID NO: 2) or a fragment thereof which is capable of treating Campylobacter spp infection;
c) a peptide comprising the amino acid sequence STALPLWSNYSYDSA (SEQ ID NO: 3) or a fragment thereof which is capable of treating Campylobacter spp infection;
d) a peptide comprising the amino acid sequence RTFGAKPPNIPFPRR (SEQ ID NO: 4) or a fragment thereof which is capable of treating Campylobacter spp infection;
e) a peptide comprising the amino acid sequence KAIAFQSSPGSPVLL (SEQ ID NO: 5) or a fragment thereof which is capable of treating Campylobacter spp infection;
f) a peptide comprising the amino acid sequence TCNLSDYTLPRARVL (SEQ ID NO: 6) or a fragment thereof which is capable of treating Campylobacter spp infection;
g) a peptide comprising the amino acid sequence NFLCPSPYPKVQPGL (SEQ ID NO: 7) or a fragment thereof which is capable of treating Campylobacter spp infection;
h) a peptide comprising the amino acid sequence VPESIHKASLVCYRF (SEQ ID NO: 8) or a fragment thereof which is capable of treating Campylobacter spp infection;
i) a peptide comprising the amino acid sequence SRPIARFYTWSQNTT (SEQ ID NO: 9) or a fragment thereof which is capable of treating Campylobacter spp infection;
j) a peptide comprising the amino acid sequence FSPFRISELVYTLHP (SEQ ID NO: 10) or a fragment thereof which is capable treating Campylobacter spp infection;
k) a peptide comprising the amino acid sequence LPFNLAKPELYIFVQ (SEQ ID NO: 11) or a fragment thereof which is capable of treating Campylobacter spp infection;
l) a peptide comprising the amino acid sequence GRFLIRVTSSPLGPD (SEQ ID NO: 12) or a fragment thereof which is capable of treating Campylobacter spp infection;
m) a peptide comprising the amino acid sequence TGSGLYLHQMVYLYQ (SEQ ID NO: 13) or a fragment thereof which is capable of treating Campylobacter spp infection;
n) a peptide comprising the amino acid sequence FLIDSPLASIGPTSM (SEQ ID NO: 14) or a fragment thereof which is capable of treating Campylobacter spp infection;
o) a peptide comprising the amino acid sequence FMIDSPLASIGPTSM (SEQ ID NO: 15) or a fragment thereof which is capable of treating Campylobacter spp infection;
p) a peptide comprising the amino acid sequence FLSDPPAPPTSPGVV (SEQ ID NO: 16) or a fragment thereof which is capable of treating Campylobacter spp infection;
q) a peptide comprising the amino acid sequence VLAWFSPLTLESSRL (SEQ ID NO: 17) or a fragment thereof which is capable of treating Campylobacter spp infection;
r) a peptide comprising the amino acid sequence VIDLSGTRKSSSGTM (SEQ ID NO: 18) or a fragment thereof which is capable of treating Campylobacter spp infection;
s) a peptide comprising the amino acid sequence VRKTTSHPPSYALLH (SEQ ID NO: 19) or a fragment thereof which is capable of treating Campylobacter spp infection;
t) a peptide comprising the amino acid sequence TPPYRAALATPVLLL (SEQ ID NO: 20) or a fragment thereof which is capable of treating Campylobacter spp infection;
u) a peptide comprising the amino acid sequence LSAPSPMFLPPVNPH (SEQ ID NO: 21) or a fragment thereof which is capable of treating Campylobacter spp infection;
v) a peptide comprising the amino acid sequence HRPVKTPANAPTTMM (SEQ ID NO: 22) or a fragment thereof which is capable of treating Campylobacter spp infection;
w) a peptide comprising the amino acid sequence CFNDPLDIVPPMLLL (SEQ ID NO: 23) or a fragment thereof which is capable of treating Campylobacter spp infection;
x) a peptide comprising the amino acid sequence AHAPCSLFFPLSLRP (SEQ ID NO: 24) or a fragment thereof which is capable of treating Campylobacter spp infection;
y) a peptide comprising the amino acid sequence KALYALHVPSMQVFA (SEQ ID NO: 25) or a fragment thereof which is capable of treating Campylobacter spp infection;
z) a peptide comprising the amino acid sequence RPSRWPWQEPLPISI (SEQ ID NO: 26) or a fragment thereof which is capable of treating Campylobacter spp infection; and
aa) a peptide comprising the amino acid sequence KVQIIPKDTLAPLPP (SEQ ID NO: 27) or a fragment thereof which is capable of treating Campylobacter spp infection in the manufacture of a medicament for the treatment of Campylobacter spp infection in a subject.

In another aspect, the present invention relates to a method of diagnosing a Campylobacter spp. infection in a subject comprising:

1) providing a sample from the subject;
2) contacting the sample with one or more peptides under conditions which permit binding of the one or more peptides to Campylobacter sp.; and
3) determining if the one or more peptides bind to a Campylobacter spp. in the sample, wherein the one or more peptides are selected from the group consisting of: a) a peptide comprising the amino acid sequence EGVLGAALSAFSFDS (SEQ ID NO: 1) or a fragment thereof which is capable of diagnosing Campylobacter spp infection; b) a peptide comprising the amino acid sequence QAQPCPNQPDGSVYA (SEQ ID NO: 2) or a fragment thereof which is capable of diagnosing Campylobacter spp infection; c) a peptide comprising the amino acid sequence STALPLWSNYSYDSA (SEQ ID NO: 3) or a fragment thereof which is capable of diagnosing Campylobacter spp infection; d) a peptide comprising the amino acid sequence RTFGAKPPNIPFPRR (SEQ ID NO: 4) or a fragment thereof which is capable of diagnosing Campylobacter spp infection; e) a peptide comprising the amino acid sequence KAIAFQSSPGSPVLL (SEQ ID NO: 5) or a fragment thereof which is capable of diagnosing Campylobacter spp infection; f) a peptide comprising the amino acid sequence TCNLSDYTLPRARVL (SEQ ID NO: 6) or a fragment thereof which is capable of diagnosing Campylobacter spp infection; g) a peptide comprising the amino acid sequence NFLCPSPYPKVQPGL (SEQ ID NO: 7) or a fragment thereof which is capable of diagnosing Campylobacter spp infection; h) a peptide comprising the amino acid sequence VPESIHKASLVCYRF (SEQ ID NO: 8) or a fragment thereof which is capable of diagnosing Campylobacter spp infection; i) a peptide comprising the amino acid sequence SRPIARFYTWSQNTT (SEQ ID NO: 9) or a fragment thereof which is capable of diagnosing Campylobacter spp infection; j) a peptide comprising the amino acid sequence FSPFRISELVYTLHP (SEQ ID NO: 10) or a fragment thereof which is capable diagnosing Campylobacter spp infection; k) a peptide comprising the amino acid sequence LPFNLAKPELYIFVQ (SEQ ID NO: 11) or a fragment thereof which is capable of diagnosing Campylobacter spp infection; 1) a peptide comprising the amino acid sequence GRFLIRVTSSPLGPD (SEQ ID NO: 12) or a fragment thereof which is capable of diagnosing Campylobacter spp infection; m) a peptide comprising the amino acid sequence TGSGLYLHQMVYLYQ (SEQ ID NO: 13) or a fragment thereof which is capable of diagnosing Campylobacter spp infection; n) a peptide comprising the amino acid sequence FLIDSPLASIGPTSM (SEQ ID NO: 14) or a fragment thereof which is capable of diagnosing Campylobacter spp infection, o) a peptide comprising the amino acid sequence FMIDSPLASIGPTSM (SEQ ID NO: 15) or a fragment thereof which is capable of diagnosing Campylobacter spp infection; p) a peptide comprising the amino acid sequence FLSDPPAPPTSPGVV (SEQ ID NO: 16) or a fragment thereof which is capable of diagnosing Campylobacter spp infection; q) a peptide comprising the amino acid sequence VLAWFSPLTLESSRL (SEQ ID NO: 17) or a fragment thereof which is capable of diagnosing Campylobacter spp infection; r) a peptide comprising the amino acid sequence VIDLSGTRKSSSGTM (SEQ ID NO: 18) or a fragment thereof which is capable of diagnosing Campylobacter spp infection; s) a peptide comprising the amino acid sequence VRKTTSHPPSYALLH (SEQ ID NO: 19) or a fragment thereof which is capable of diagnosing Campylobacter spp infection; t) a peptide comprising the amino acid sequence TPPYRAALATPVLLL (SEQ ID NO: 20) or a fragment thereof which is capable of diagnosing Campylobacter spp infection; u) a peptide comprising the amino acid sequence LSAPSPMFLPPVNPH (SEQ ID NO: 21) or a fragment thereof which is capable of diagnosing Campylobacter spp infection; v) a peptide comprising the amino acid sequence HRPVKTPANAPTTMM (SEQ ID NO: 22) or a fragment thereof which is capable of diagnosing Campylobacter spp infection; w) a peptide comprising the amino acid sequence CFNDPLDIVPPMLLL (SEQ ID NO: 23) or a fragment thereof which is capable of diagnosing Campylobacter spp infection; x) a peptide comprising the amino acid sequence AHAPCSLFFPLSLRP (SEQ ID NO: 24) or a fragment thereof which is capable of diagnosing Campylobacter spp infection; y) a peptide comprising the amino acid sequence KALYALHVPSMQVFA (SEQ ID NO: 25) or a fragment thereof which is capable of diagnosing Campylobacter spp infection; z) a peptide comprising the amino acid sequence RPSRWPWQEPLPISI (SEQ ID NO: 26) or a fragment thereof which is capable of diagnosing Campylobacter spp infection; and aa) a peptide comprising the amino acid sequence KVQIIPKDTLAPLPP (SEQ ID NO: 27) or a fragment thereof which is capable of diagnosing Campylobacter spp infection.

In one embodiment, the one or more peptides bind to the Campylobacter spp. to prevent, treat or diagnose Campylobacter spp. infection. The one or more peptides may bind specifically to the Campylobacter species to prevent, treat or diagnose Campylobacter spp. infection.

The Campylobacter spp. may be any species of Campylobacter. In one embodiment, the Campylobacter spp. is Campylobacter jejuni. In another embodiment, the Campylobacter spp. is selected from the group consisting of Campylobacter coli, C. lari, C. Upsaliensis, C. concisus, C. curvus, C. fetus, C. graciius, C. helveticus, C. hyoilei, C. hyointestinalis, C. lanienae, C. mucosalis, C. rectus, C. showae, and C. sputorum.

The subject may be human. The subject may be poultry, such as a chicken, duck, goose or turkey. The subject may be a domesticated animal, for example a cat or dog, or an agricultural animal, for example a pig, cow or sheep.

In one embodiment, the one or more peptides is a peptide comprising the amino acid sequence EGVLGAALSAFSFDS (SEQ ID NO: 1), or a fragment thereof which is capable of preventing, treating or diagnosing Campylobacter spp infection. In one embodiment, the one or more peptides is a peptide comprising the amino acid sequence QAQPCPNQPDGSVYA (SEQ ID NO: 2) or a fragment thereof which is capable of preventing, treating or diagnosing Campylobacter spp infection. In one embodiment, the one or more peptides is a peptide comprising the amino acid sequence STALPLWSNYSYDSA (SEQ ID NO: 3) or a fragment thereof which is capable of preventing, treating or diagnosing Campylobacter spp infection. In one embodiment, the one or more peptides is a peptide comprising the amino acid sequence RTFGAKPPNIPFPRR (SEQ ID NO: 4) or a fragment thereof which is capable of preventing, treating or diagnosing Campylobacter spp infection. In one embodiment, the one or more peptides is a peptide comprising the amino acid sequence KAIAFQSSPGSPVLL (SEQ ID NO: 5) or a fragment thereof which is capable of preventing, treating or diagnosing Campylobacter spp infection. In one embodiment, the one or more peptides is a peptide comprising the amino acid sequence TCNLSDYTLPRARVL (SEQ ID NO: 6) or a fragment thereof which is capable of preventing, treating or diagnosing Campylobacter spp infection. In one embodiment, the one or more peptides is a peptide comprising the amino acid sequence NFLCPSPYPKVQPGL (SEQ ID NO: 7) or a fragment thereof which is capable of preventing, treating or diagnosing Campylobacter spp infection. In one embodiment, the one or more peptides is a peptide comprising the amino acid sequence VPESIHKASLVCYRF (SEQ ID NO: 8) or a fragment thereof which is capable of preventing, treating or diagnosing Campylobacter spp infection. In one embodiment, the one or more peptides is a peptide comprising the amino acid sequence SRPIARFYTWSQNTT (SEQ ID NO: 9) or a fragment thereof which is capable of preventing, treating or diagnosing Campylobacter spp infection. In one embodiment, the one or more peptides is a peptide comprising the amino acid sequence FSPFRISELVYTLHP (SEQ ID NO: 10) or a fragment thereof which is capable of preventing, treating or diagnosing Campylobacter spp infection. In one embodiment, the one or more peptides is a peptide comprising the amino acid sequence LPFNLAKPELYIFVQ (SEQ ID NO: 11) or a fragment thereof which is capable of preventing, treating or diagnosing Campylobacter spp infection. In one embodiment, the one or more peptides is a peptide comprising the amino acid sequence GRFLIRVTSSPLGPD (SEQ ID NO: 12) or a fragment thereof which is capable of preventing, treating or diagnosing Campylobacter spp infection. In one embodiment, the one or more peptides is a peptide comprising the amino acid sequence TGSGLYLHQMVYLYQ (SEQ ID NO: 13) or a fragment thereof which is capable of preventing, treating or diagnosing Campylobacter spp infection. In one embodiment, the one or more peptides is a peptide comprising the amino acid sequence FLIDSPLASIGPTSM (SEQ ID NO: 14) or a fragment thereof which is capable of preventing, treating or diagnosing Campylobacter spp infection. In one embodiment, the one or more peptides is a peptide comprising the amino acid sequence FMIDSPLASIGPTSM (SEQ ID NO: 15) or a fragment thereof which is capable of preventing, treating or diagnosing Campylobacter spp infection. In one embodiment, the one or more peptides is a peptide comprising the amino acid sequence FLSDPPAPPTSPGVV (SEQ ID NO: 16), or a fragment thereof which is capable of preventing, treating or diagnosing Campylobacter spp infection. In one embodiment, the one or more peptides is a peptide comprising the amino acid sequence VLAWFSPLTLESSRL (SEQ ID NO: 17) or a fragment thereof which is capable of preventing, treating or diagnosing Campylobacter spp infection. In one embodiment, the one or more peptides is a peptide comprising the amino acid sequence VIDLSGTRKSSSGTM (SEQ ID NO: 18) or a fragment thereof which is capable of preventing, treating or diagnosing Campylobacter spp infection. In one embodiment, the one or more peptides is a peptide comprising the amino acid sequence VRKTTSHPPSYALLH (SEQ ID NO: 19) or a fragment thereof which is capable of preventing, treating or diagnosing Campylobacter spp infection. In one embodiment, the one or more peptides is a peptide comprising the amino acid sequence TPPYRAALATPVLLL (SEQ ID NO: 20) or a fragment thereof which is capable of preventing, treating or diagnosing Campylobacter spp infection. In one embodiment, the one or more peptides is a peptide comprising the amino acid sequence LSAPSPMFLPPVNPH (SEQ ID NO: 21) or a fragment thereof which is capable of preventing, treating or diagnosing Campylobacter spp infection. In one embodiment, the one or more peptides is a peptide comprising the amino acid sequence HRPVKTPANAPTTMM (SEQ ID NO: 22) or a fragment thereof which is capable of preventing, treating or diagnosing Campylobacter spp infection. In one embodiment, the one or more peptides is a peptide comprising the amino acid sequence CFNDPLDIVPPMLLL (SEQ ID NO: 23) or a fragment thereof which is capable of preventing, treating or diagnosing Campylobacter spp infection. In one embodiment, the one or more peptides is a peptide comprising the amino acid sequence AHAPCSLFFPLSLRP (SEQ ID NO: 24) or a fragment thereof which is capable of preventing, treating or diagnosing Campylobacter spp infection. In one embodiment, the one or more peptides is a peptide comprising the amino acid sequence KALYALHVPSMQVFA (SEQ ID NO: 25) or a fragment thereof which is capable of preventing, treating or diagnosing Campylobacter spp infection. In one embodiment, the one or more peptides is a peptide comprising the amino acid sequence RPSRWPWQEPLPISI (SEQ ID NO: 26) or a fragment thereof which is capable of preventing, treating or diagnosing Campylobacter spp infection. In one embodiment, the one or more peptides is a peptide comprising the amino acid sequence KVQIIPKDTLAPLPP (SEQ ID NO: 27) or a fragment thereof which is capable of preventing, treating or diagnosing Campylobacter spp infection.

In another aspect, the present invention relates to an isolated peptide comprising an amino acid sequence selected from the group consisting of:

| | |
|---|---|
| EGVLGAALSAFSFDS; | (SEQ ID NO: 1) |
| QAQPCPNQPDGSVYA; | (SEQ ID NO: 2) |
| STALPLWSNYSYDSA; | (SEQ ID NO: 3) |
| RTFGAKPPNIPFPRR; | (SEQ ID NO: 4) |
| KAIAFQSSPGSPVLL; | (SEQ ID NO: 5) |
| TCNLSDYTLPRARVL; | (SEQ ID NO: 6) |
| NFLCPSPYPKVQPGL; | (SEQ ID NO: 7) |
| VPESIHKASLVCYRF; | (SEQ ID NO: 8) |
| SRPIARFYTWSQNTT; | (SEQ ID NO: 9) |
| FSPFRISELVYTLHP; | (SEQ ID NO: 10) |
| LPFNLAKPELYIFVQ; | (SEQ ID NO: 11) |
| GRFLIRVTSSPLGPD; | (SEQ ID NO: 12) |
| TGSGLYLHQMVYLYQ; | (SEQ ID NO: 13) |
| FLIDSPLASIGPTSM, | (SEQ ID NO: 14) |
| FMIDSPLASIGPTSM; | (SEQ ID NO: 15) |
| FLSDPPAPPTSPGVV; | (SEQ ID NO: 16) |
| VLAWFSPLTLESSRL; | (SEQ ID NO: 17) |
| VIDLSGTRKSSSGTM; | (SEQ ID NO: 18) |
| VRKTTSHPPSYALLH; | (SEQ ID NO: 19) |
| TPPYRAALATPVLLL; | (SEQ ID NO: 20) |
| LSAPSPMFLPPVNPH; | (SEQ ID NO: 21) |
| HRPVKTPANAPTTMM; | (SEQ ID NO: 22) |
| CFNDPLDIVPPMLLL; | (SEQ ID NO: 23) |
| AHAPCSLFFPLSLRP; | (SEQ ID NO: 24) |
| KALYALHVPSMQVFA; | (SEQ ID NO: 25) |
| RPSRWPWQEPLPISI; | (SEQ ID NO: 26) |
| and | |
| KVQIIPKDTLAPLPP. | (SEQ ID NO: 27) |

In another aspect, the present invention relates to an isolated peptide of 15 to 100 amino acid residues comprising an amino acid sequence selected from the group consisting of: EGVLGAALSAFSFDS (SEQ ID NO: 1); QAQPCPNQPDGSVYA (SEQ ID NO: 2); STALPLWSNYSYDSA (SEQ ID NO: 3); RTFGAKPPNIPFPRR (SEQ ID NO: 4); KAIAFQSSPGSPVLL (SEQ ID NO: 5); TCNLSDYTLPRARVL (SEQ ID NO: 6); NFLCPSPYPKVQPGL (SEQ ID NO: 7); VPESIHKASLVCYRF (SEQ ID NO: 8); SRPIARFYTWSQNTT (SEQ ID NO: 9); FSPFRISELVYTLHP (SEQ ID NO: 10); LPFNLAKPELYIFVQ (SEQ ID NO: 11); GRFLIRVTSSPLGPD (SEQ ID NO: 12); TGSGLYLHQMVYLYQ (SEQ ID NO: 13); FLIDSPLASIGPTSM (SEQ ID NO: 14), FMIDSPLASIGPTSM (SEQ ID NO: 15); FLSDPPAPPTSPGVV (SEQ ID NO: 16); VLAWFSPLTLESSRL (SEQ ID NO: 17); VIDLSGTRKSSSGTM (SEQ ID NO: 18); VRKTTSHPPSYALLH (SEQ ID NO: 19); TPPYRAALATPVLLL (SEQ ID NO: 20); LSAPSPMFLPPVNPH (SEQ ID NO: 21); HRPVKTPANAPTTMM (SEQ ID NO: 22); CFNDPLDIVPPMLLL (SEQ ID NO: 23); AHAPCSLFFPLSLRP (SEQ ID NO: 24); KALYALHVPSMQVFA (SEQ ID NO: 25); RPSRWPWQEPLPISI (SEQ ID NO: 26); and KVQIIPKDTLAPLPP (SEQ ID NO: 27).

In another aspect, the present invention relates to a synthetic peptide comprising an amino acid sequence selected from the group consisting of:

| | |
|---|---|
| EGVLGAALSAFSFDS; | (SEQ ID NO: 1) |
| QAQPCPNQPDGSVYA; | (SEQ ID NO: 2) |
| STALPLWSNYSYDSA; | (SEQ ID NO: 3) |
| RTFGAKPPNIPFPRR; | (SEQ ID NO: 4) |
| KAIAFQSSPGSPVLL; | (SEQ ID NO: 5) |
| TCNLSDYTLPRARVL; | (SEQ ID NO: 6) |
| NFLCPSPYPKVQPGL; | (SEQ ID NO: 7) |
| VPESIHKASLVCYRF; | (SEQ ID NO: 8) |
| SRPIARFYTWSQNTT; | (SEQ ID NO: 9) |
| FSPFRISELVYTLHP; | (SEQ ID NO: 10) |
| LPFNLAKPELYIFVQ; | (SEQ ID NO: 11) |
| GRFLIRVTSSPLGPD; | (SEQ ID NO: 12) |
| TGSGLYLHQMVYLYQ; | (SEQ ID NO: 13) |
| FLIDSPLASIGPTSM, | (SEQ ID NO: 14) |
| FMIDSPLASIGPTSM; | (SEQ ID NO: 15) |
| FLSDPPAPPTSPGVV; | (SEQ ID NO: 16) |
| VLAWFSPLTLESSRL; | (SEQ ID NO: 17) |
| VIDLSGTRKSSSGTM; | (SEQ ID NO: 18) |
| VRKTTSHPPSYALLH; | (SEQ ID NO: 19) |
| TPPYRAALATPVLLL; | (SEQ ID NO: 20) |
| LSAPSPMFLPPVNPH; | (SEQ ID NO: 21) |
| HRPVKTPANAPTTMM; | (SEQ ID NO: 22) |
| CFNDPLDIVPPMLLL; | (SEQ ID NO: 23) |
| AHAPCSLFFPLSLRP; | (SEQ ID NO: 24) |
| KALYALHVPSMQVFA; | (SEQ ID NO: 25) |
| RPSRWPWQEPLPISI; | (SEQ ID NO: 26) |
| and | |
| KVQIIPKDTLAPLPP. | (SEQ ID NO: 27) |

In another aspect, the present invention relates to a fusion protein comprising one or more peptides, wherein the one or more peptides are selected from the group consisting of: EGVLGAALSAFSFDS (SEQ ID NO: 1); QAQPCPNQPDGSVYA (SEQ ID NO: 2); STALPLWSNYSYDSA (SEQ ID NO: 3); RTFGAKPPNIPFPRR (SEQ ID NO: 4); KAIAFQSSPGSPVLL (SEQ ID NO: 5); TCNLSDYTLPRARVL (SEQ ID NO: 6); NFLCPSPYPKVQPGL (SEQ ID NO: 7); VPESIHKASLVCYRF (SEQ ID NO: 8); SRPIARFYTWSQNTT (SEQ ID NO: 9); FSPFRISELVYTLHP (SEQ ID NO: 10); LPFNLAKPELYIFVQ (SEQ ID NO: 11); GRFLIRVTSSPLGPD (SEQ ID NO: 12); TGSGLYLHQMVYLYQ (SEQ ID NO: 13); FLIDSPLASIGPTSM (SEQ ID NO: 14), FMIDSPLASIGPTSM (SEQ ID NO: 15); FLSDPPAPPTSPGVV (SEQ ID NO: 16); VLAWFSPLTLESSRL (SEQ ID NO: 17); VIDLSGTRKSSSGTM (SEQ ID NO: 18); VRKTTSHPPSYALLH (SEQ ID NO: 19); TPPYRAALATPVLLL (SEQ ID NO: 20); LSAPSPMFLPPVNPH (SEQ ID NO: 21); HRPVKTPANAPTTMM (SEQ ID NO: 22); CFNDPLDIVPPMLLL (SEQ ID NO: 23); AHAPCSLFFPLSLRP (SEQ ID NO: 24); KALYALHVPSMQVFA (SEQ ID NO: 25); RPSRWPWQEPLPISI (SEQ ID NO: 26); and KVQIIPKDTLAPLPP (SEQ ID NO: 27).

In one embodiment, the fusion protein further comprises a phage coat or part thereof.

In another aspect, the present invention relates to fd-tet phage comprising one or more peptides, wherein the one or more peptides are selected from the group consisting of: EGVLGAALSAFSFDS (SEQ ID NO: 1); QAQPCPNQPDGSVYA (SEQ ID NO: 2); STALPLWSNYSYDSA (SEQ ID NO: 3); RTFGAKPPNIPFPRR (SEQ ID NO: 4); KAIAFQSSPGSPVLL (SEQ ID NO: 5); TCNLSDYTLPRARVL (SEQ ID NO: 6); NFLCPSPYPKVQPGL (SEQ ID NO: 7); VPESIHKASLVCYRF (SEQ ID NO: 8); SRPIARFYTWSQNTT (SEQ ID NO: 9); FSPFRISELVYTLHP (SEQ ID NO: 10); LPFNLAKPELYIFVQ (SEQ ID NO: 11); GRFLIRVTSSPLGPD (SEQ ID NO: 12); TGSGLYLHQMVYLYQ (SEQ ID NO: 13); FLIDSPLASIGPTSM (SEQ ID NO: 14), FMIDSPLASIGPTSM (SEQ ID NO: 15); FLSDPPAPPTSPGVV (SEQ ID NO: 16); VLAWFSPLTLESSRL (SEQ ID NO: 17); VIDLSGTRKSSSGTM (SEQ ID NO: 18); VRKTTSHPPSYALLH (SEQ ID NO: 19); TPPYRAALATPVLLL (SEQ ID NO: 20); LSAPSPMFLPPVNPH (SEQ ID NO: 21); HRPVKTPANAPTTMM (SEQ ID NO: 22); CFNDPLDIVPPMLLL (SEQ ID NO: 23); AHAPCSLFFPLSLRP (SEQ ID NO: 24); KALYALHVPSMQVFA (SEQ ID NO: 25); RPSRWPWQEPLPISI (SEQ ID NO: 26); and KVQIIPKDTLAPLPP (SEQ ID NO: 27).

In another aspect, the present invention relates to a composition comprising one or more peptides, wherein the one of more peptides are selected from the group consisting of: a) a peptide comprising the amino acid sequence EGVLGAALSAFSFDS (SEQ ID NO: 1); b) a peptide comprising the amino acid sequence QAQPCPNQPDGSVYA (SEQ ID NO: 2); c) a peptide comprising the amino acid sequence STALPLWSNYSYDSA (SEQ ID NO: 3); d) a peptide comprising the amino acid sequence RTFGAKPPNIPFPRR (SEQ ID NO: 4); e) a peptide comprising the amino acid sequence KAIAFQSSPGSPVLL (SEQ ID NO: 5); f) a peptide comprising the amino acid sequence TCNLSDYTLPRARVL (SEQ ID NO: 6); g) a peptide comprising the amino acid sequence NFLCPSPYPKVQPGL (SEQ ID NO: 7); h) a peptide comprising the amino acid sequence VPESIHKASLVCYRF (SEQ ID NO: 8); i) a peptide comprising the amino acid sequence SRPIARFYTWSQNTT (SEQ ID NO: 9); j) a peptide comprising the amino acid sequence FSPFRISELVYTLHP (SEQ ID NO: 10); k) a peptide comprising the amino acid sequence LPFNLAKPELYIFVQ (SEQ ID NO: 11); 1) a peptide comprising the amino acid sequence GRFLIRVTSSPLGPD (SEQ ID NO: 12; m) a peptide comprising the amino acid sequence TGSGLYLHQMVYLYQ (SEQ ID NO: 13); n) a peptide comprising the amino acid sequence FLIDSPLASIGPTSM (SEQ ID NO: 14), o) a peptide comprising the amino acid sequence FMIDSPLASIGPTSM (SEQ ID NO: 15); p) a peptide comprising the amino acid sequence FLSDPPAPPTSPGVV (SEQ ID NO: 16); q) a peptide comprising the amino acid sequence VLAWFSPLTLESSRL (SEQ ID NO: 17); r) a peptide comprising the amino acid sequence VIDLSGTRKSSSGTM (SEQ ID NO: 18); s) a peptide comprising the amino acid sequence VRKTTSHPPSYALLH (SEQ ID NO: 19); t) a peptide comprising the amino acid sequence TPPYRAALATPVLLL (SEQ ID NO: 20); u) a peptide comprising the amino acid sequence LSAPSPMFLPPVNPH (SEQ ID NO: 21); v) a peptide comprising the amino acid sequence HRPVKTPANAPTTMM (SEQ ID NO: 22); w) a peptide comprising the amino acid sequence CFNDPLDIVPPMLLL (SEQ ID NO: 23); x) a peptide comprising the amino acid sequence AHAPCSLFFPLSLRP (SEQ ID NO: 24); y) a peptide comprising the amino acid sequence KALYALHVPSMQVFA (SEQ ID NO: 25); z) a peptide comprising the amino acid sequence RPSRWPWQEPLPISI (SEQ ID NO: 26); and aa) a peptide comprising the amino acid sequence KVQIIPKDTLAPLPP (SEQ ID NO: 27).

In another aspect, the present invention relates to a pharmaceutical composition comprising one or more peptides and a pharmaceutically acceptable carrier, wherein the one of more peptides are selected from the group consisting of: a) a peptide comprising the amino acid sequence EGVLGAALSAFSFDS (SEQ ID NO: 1); b) a peptide comprising the amino acid sequence QAQPCPNQPDGSVYA (SEQ ID NO: 2); c) a peptide comprising the amino acid sequence STALPLWSNYSYDSA (SEQ ID NO: 3);

d) a peptide comprising the amino acid sequence RTFGAKPPNIPFPRR (SEQ ID NO: 4); e) a peptide comprising the amino acid sequence KAIAFQSSPGSPVLL (SEQ ID NO: 5); f) a peptide comprising the amino acid sequence TCNLSDYTLPRARVL (SEQ ID NO: 6); g) a peptide comprising the amino acid sequence NFLCPSPYPKVQPGL (SEQ ID NO: 7); h) a peptide comprising the amino acid sequence VPESIHKASLVCYRF (SEQ ID NO: 8); i) a peptide comprising the amino acid sequence SRPIARFYTWSQNTT (SEQ ID NO: 9); j) a peptide comprising the amino acid sequence FSPFRISELVYTLHP (SEQ ID NO: 10); k) a peptide comprising the amino acid sequence LPFNLAKPELYIFVQ (SEQ ID NO: 11); 1) a peptide comprising the amino acid sequence GRFLIRVTSSPLGPD (SEQ ID NO: 12; m) a peptide comprising the amino acid sequence TGSGLYLHQMVYLYQ (SEQ ID NO: 13); n) a peptide comprising the amino acid sequence FLIDSPLASIGPTSM (SEQ ID NO: 14), o) a peptide comprising the amino acid sequence FMIDSPLASIGPTSM (SEQ ID NO: 15); p) a peptide comprising the amino acid sequence FLSDPPAPPTSPGVV (SEQ ID NO: 16); q) a peptide comprising the amino acid sequence VLAWFSPLTLESSRL (SEQ ID NO: 17); r) a peptide comprising the amino acid sequence VIDLSGTRKSSSGTM (SEQ ID NO: 18); s) a peptide comprising the amino acid sequence VRKTTSHPPSYALLH (SEQ ID NO: 19); t) a peptide comprising the amino acid sequence TPPYRAALATPVLLL (SEQ ID NO: 20); u) a peptide comprising the amino acid sequence LSAPSPMFLPPVNPH (SEQ ID NO: 21); v) a peptide comprising the amino acid sequence HRPVKTPANAPTTMM (SEQ ID NO: 22); w) a peptide comprising the amino acid sequence CFNDPLDIVPPMLLL (SEQ ID NO: 23); x) a peptide comprising the amino acid sequence AHAPCSLFFPLSLRP (SEQ ID NO: 24); y) a peptide comprising the amino acid sequence KALYALHVPSMQVFA (SEQ ID NO: 25); z) a peptide comprising the amino acid sequence RPSRWPWQEPLPISI (SEQ ID NO: 26); and aa) a peptide comprising the amino acid sequence KVQIIPKDTLAPLPP (SEQ ID NO: 27).

In another aspect, the present invention relates to an isolated nucleic acid molecule which encodes a peptide selected from the group consisting of: a) a peptide comprising the amino acid sequence EGVLGAALSAFSFDS (SEQ ID NO: 1); b) a peptide comprising the amino acid sequence QAQPCPNQPDGSVYA (SEQ ID NO: 2); c) a peptide comprising the amino acid sequence STALPLWSNYSYDSA (SEQ ID NO: 3); d) a peptide comprising the amino acid sequence RTFGAKPPNIPFPRR (SEQ ID NO: 4); e) a peptide comprising the amino acid sequence KAIAFQSSPGSPVLL (SEQ ID NO: 5); f) a peptide comprising the amino acid sequence TCNLSDYTLPRARVL (SEQ ID NO: 6); g) a peptide comprising the amino acid sequence NFLCPSPYPKVQPGL (SEQ ID NO: 7); h) a peptide comprising the amino acid sequence VPESIHKASLVCYRF (SEQ ID NO: 8); i) a peptide comprising the amino acid sequence SRPIARFYTWSQNTT (SEQ ID NO: 9); j) a peptide comprising the amino acid sequence FSPFRISELVYTLHP (SEQ ID NO: 10); k) a peptide comprising the amino acid sequence LPFNLAKPELYIFVQ (SEQ ID NO: 11); 1) a peptide comprising the amino acid sequence GRFLIRVTSSPLGPD (SEQ ID NO: 12); m) a peptide comprising the amino acid sequence TGSGLYLHQMVYLYQ (SEQ ID NO: 13); n) a peptide comprising the amino acid sequence FLIDSPLASIGPTSM (SEQ ID NO: 14), o) a peptide comprising the amino acid sequence FMIDSPLASIGPTSM (SEQ ID NO: 15); p) a peptide comprising the amino acid sequence FLSDPPAPPTSPGVV (SEQ ID NO: 16); q) a peptide comprising the amino acid sequence VLAWFSPLTLESSRL (SEQ ID NO: 17); r) a peptide comprising the amino acid sequence VIDLSGTRKSSSGTM (SEQ ID NO: 18); s) a peptide comprising the amino acid sequence VRKTTSHPPSYALLH (SEQ ID NO: 19); t) a peptide comprising the amino acid sequence TPPYRAALATPVLLL (SEQ ID NO: 20); u) a peptide comprising the amino acid sequence LSAPSPMFLPPVNPH (SEQ ID NO: 21); v) a peptide comprising the amino acid sequence HRPVKTPANAPTTMM (SEQ ID NO: 22); w) a peptide comprising the amino acid sequence CFNDPLDIVPPMLLL (SEQ ID NO: 23); x) a peptide comprising the amino acid sequence AHAPCSLFFPLSLRP (SEQ ID NO: 24); y) a peptide comprising the amino acid sequence KALYALHVPSMQVFA (SEQ ID NO: 25); z) a peptide comprising the amino acid sequence RPSRWPWQEPLPISI (SEQ ID NO: 26); and aa) a peptide comprising the amino acid sequence KVQIIPKDTLAPLPP (SEQ ID NO: 27).

In another aspect, the present invention relates to a recombinant vector comprising an isolated nucleic acid molecule which encodes a peptide selected from the group consisting of: a) a peptide comprising the amino acid sequence EGVLGAALSAFSFDS (SEQ ID NO: 1); b) a peptide comprising the amino acid sequence QAQPCPNQPDGSVYA (SEQ ID NO: 2); c) a peptide comprising the amino acid sequence STALPLWSNYSYDSA (SEQ ID NO: 3); d) a peptide comprising the amino acid sequence RTFGAKPPNIPFPRR (SEQ ID NO: 4); e) a peptide comprising the amino acid sequence KAIAFQSSPGSPVLL (SEQ ID NO: 5); f) a peptide comprising the amino acid sequence TCNLSDYTLPRARVL (SEQ ID NO: 6); g) a peptide comprising the amino acid sequence NFLCPSPYPKVQPGL (SEQ ID NO: 7); h) a peptide comprising the amino acid sequence VPESIHKASLVCYRF (SEQ ID NO: 8); i) a peptide comprising the amino acid sequence SRPIARFYTWSQNTT (SEQ ID NO: 9); j) a peptide comprising the amino acid sequence FSPFRISELVYTLHP (SEQ ID NO: 10); k) a peptide comprising the amino acid sequence LPFNLAKPELYIFVQ (SEQ ID NO: 11); 1) a peptide comprising the amino acid sequence GRFLIRVTSSPLGPD (SEQ ID NO: 12); m) a peptide comprising the amino acid sequence TGSGLYLHQMVYLYQ (SEQ ID NO: 13); n) a peptide comprising the amino acid sequence FLIDSPLASIGPTSM (SEQ ID NO: 14), o) a peptide comprising the amino acid sequence FMIDSPLASIGPTSM (SEQ ID NO: 15); p) a peptide comprising the amino acid sequence FLSDPPAPPTSPGVV (SEQ ID NO: 16); q) a peptide comprising the amino acid sequence VLAWFSPLTLESSRL (SEQ ID NO: 17); r) a peptide comprising the amino acid sequence VIDLSGTRKSSSGTM (SEQ ID NO: 18); s) a peptide comprising the amino acid sequence VRKTTSHPPSYALLH (SEQ ID NO: 19); t) a peptide comprising the amino acid sequence TPPYRAALATPVLLL (SEQ ID NO: 20); u) a peptide comprising the amino acid sequence LSAPSPMFLPPVNPH (SEQ ID NO: 21); v) a peptide comprising the amino acid sequence HRPVKTPANAPTTMM (SEQ ID NO: 22); w) a peptide comprising the amino acid sequence CFNDPLDIVPPMLLL (SEQ ID NO: 23); x) a peptide comprising the amino acid sequence AHAPCSLFFPLSLRP (SEQ ID NO: 24); y) a peptide comprising the amino acid sequence KALYALHVPSMQVFA (SEQ ID NO: 25); z) a peptide comprising the amino acid sequence RPSRWPWQEPLPISI (SEQ ID NO: 26); and aa) a peptide comprising the amino acid sequence KVQIIPKDTLAPLPP (SEQ ID NO: 27).

### BRIEF DESCRIPTION OF THE FIGURES

FIG. 1 illustrates a selection strategy for isolating phage peptides which bind to C. jejuni.
A. Phage peptides from an f88-4/15-mer library were directly selected against C. jejuni strain ACM 3393, resulting in “AP” and “AT” pools of phage peptides.
B. A phage-display library was subtracted once against C. jejuni strain ACM 3393 (black box), prior to three rounds of affinity selection against C. jejuni strain ACM 3393. This resulted in “BP” and “BT” phage peptides.
C. A phage-display library was subtracted twice against C. jejuni strain ACM 3393, prior to three rounds of affinity selection against C. jejuni strain C338 (poultry strain). This resulted in “CP” and “CT” phage peptides.
D. A phage-display library was subtracted twice against C. jejuni strain ACM 3393 and once against C. jejuni strain C338, prior to three rounds of affinity selection against C338. This resulted in “DP” and “DT” phage peptides.

FIG. 2 illustrates the different morphologies of C. jejuni strain C338 when incubated with selected phage-displayed peptides.
A. Strain C338 incubated with phage peptide CP3/1. The morphology has changed to thread-shaped.
B. Strain C338 incubated with unselected phage library showing the normal spiral-shaped morphology
C. Strain C338 incubated with BP3/5 showing coccoid shaped bacteria

FIG. 3 illustrates the amino acid alignment of AT3/5 with related proteins. The amino acid sequences deduced from AT3/5, Oryctolagus cuniculus claudin 1 (Rabbit; Accession No. ABK59974), Homo sapiens claudin 1 (Accession No. AAP97236) and Macaca mulatta (Rhesus monkey; Accession No. XP_{—}001093143) were aligned using ClustalW. GeneDoc was used as the shading editor. The shading levels used to indicate degree of conservation in each column was black for 100% conservation and grey for 75% amino acid conservation. Note that the first 120 amino acids of the amino acid alignment are presented.

FIG. 4 illustrates the amino acid alignment of BT3/3 with C. jejuni flagella hook length control proteins.

The amino acid sequences deduced from BT3/3, C. jejuni subsp. doyleii flgE gene (Accession No. EAU02210), C. jejuni isolate 5226 (Accession No. AAP34261), C. jejuni subsp. jejuni 81-176 (Accession No. CAA12127) and C. jejuni strain NCTC11168 were aligned using ClustalW with GeneDoc used as the shading editor.

### DETAILED DESCRIPTION OF THE INVENTION

### 1. Peptides

The present invention relates to isolated peptides which bind to Campylobacter spp and typically inhibit growth of or kill Campylobacter spp. The peptides are selected from the group consisting of a) a peptide comprising the amino acid sequence EGVLGAALSAFSFDS (SEQ ID NO: 1); b) a peptide comprising the amino acid sequence QAQPCPNQPDGSVYA (SEQ ID NO: 2); c) a peptide comprising the amino acid sequence STALPLWSNYSYDSA (SEQ ID NO: 3); d) a peptide comprising the amino acid sequence RTFGAKPPNIPFPRR (SEQ ID NO: 4); e) a peptide comprising the amino acid sequence KAIAFQSSPGSPVLL (SEQ ID NO: 5); f) a peptide comprising the amino acid sequence TCNLSDYTLPRARVL (SEQ ID NO: 6); g) a peptide comprising the amino acid sequence NFLCPSPYPKVQPGL (SEQ ID NO: 7); h) a peptide comprising the amino acid sequence VPESIHKASLVCYRF (SEQ ID NO: 8); i) a peptide comprising the amino acid sequence SRPIARFYTWSQNTT (SEQ ID NO: 9); j) a peptide comprising the amino acid sequence FSPFRISELVYTLHP (SEQ ID NO: 10); k) a peptide comprising the amino acid sequence LPFNLAKPELYIFVQ (SEQ ID NO: 11); 1) a peptide comprising the amino acid sequence GRFLIRVTSSPLGPD (SEQ ID NO: 12; m) a peptide comprising the amino acid sequence TGSGLYLHQMVYLYQ (SEQ ID NO: 13); n) a peptide comprising the amino acid sequence FLIDSPLASIGPTSM (SEQ ID NO: 14), o) a peptide comprising the amino acid sequence FMIDSPLASIGPTSM (SEQ ID NO: 15); p) a peptide comprising the amino acid sequence FLSDPPAPPTSPGVV (SEQ ID NO: 16); q) a peptide comprising the amino acid sequence VLAWFSPLTLESSRL (SEQ ID NO: 17); r) a peptide comprising the amino acid sequence VIDLSGTRKSSSGTM (SEQ ID NO: 18); s) a peptide comprising the amino acid sequence VRKTTSHPPSYALLH (SEQ ID NO: 19); t) a peptide comprising the amino acid sequence TPPYRAALATPVLLL (SEQ ID NO: 20); u) a peptide comprising the amino acid sequence LSAPSPMFLPPVNPH (SEQ ID NO: 21); v) a peptide comprising the amino acid sequence HRPVKTPANAPTTMM (SEQ ID NO: 22); w) a peptide comprising the amino acid sequence CFNDPLDIVPPMLLL (SEQ ID NO: 23); x) a peptide comprising the amino acid sequence AHAPCSLFFPLSLRP (SEQ ID NO: 24); y) a peptide comprising the amino acid sequence KALYALHVPSMQVFA (SEQ ID NO: 25); z) a peptide comprising the amino acid sequence RPSRWPWQEPLPISI (SEQ ID NO: 26); and aa) a peptide comprising the amino acid sequence KVQIIPKDTLAPLPP (SEQ ID NO: 27).

As described herein peptides which bind to Campylobacter jejuni were isolated from a phage-display library consisting of filamentous fd-tet phage displaying random 15-mer peptides fused to 150-300 copies of major coat protein pVIII (Scott & Smith, 1990) and contained a complexity of 2×10⁹ peptidevariants/mL [supplied by Dr. George Smith (University of Missouri-Columbia, USA)].

The peptides of the invention may be produced by recombinant methods, or may be chemically synthesized. Methods for producing recombinant peptides are known in the art and are described in, for example, Zwick et al. 1998. Methods of chemical synthesis of the peptides are known in the art and are described in, for example, they may be prepared by solid-phase synthesis as described by Stewart et al, 1984.

As described herein, the peptides of the invention bind to C. jejuni strain ACM 3393. The inventors envisage that the peptides of the invention will bind to Campylobacter spp. The peptides may exhibit preferential binding to Campylobacter spp. The peptides may bind specifically to Campylobacter spp.

The present invention also relates to fragments of the abovementioned peptides which are capable of binding to Campylobacter spp. The methods employed to produce the peptides described herein may also be used to produce fragments of the peptides which bind to Campylobacter spp.

Functional variants of the abovementioned peptides which may be capable of binding to Campylobacter spp. are also within the scope of the invention. Methods of producing functional variants of peptides are known in the art. Also encompassed in the invention are isolated peptides of 15 to about 100 amino acid residues comprising the abovementioned peptides. Examples of the peptides includes a peptide between 15 and 75 amino acid residues in length, a peptide between 15 and 50 amino acid residues in length, a peptide between 15 and 25 amino acid residues in length.

The peptides and fragments thereof may be tested against strains of Campylobacter spp as described herein for their binding ability to Campylobacter spp and/or their ability to inhibit growth of or kill Campylobacter spp.

### 2. Methods of Inhibiting Growth of or Killing Campylobacter spp

The inventors have found that peptides which bind to Campylobacter jejuni may be suitable for inhibiting growth of or killing Campylobacter spp. Accordingly, the present invention further relates to methods of inhibiting growth of or killing Campylobacter spp using one or more peptides selected from the group consisting of a) a peptide comprising the amino acid sequence EGVLGAALSAFSFDS (SEQ ID NO: 1); b) a peptide comprising the amino acid sequence QAQPCPNQPDGSVYA (SEQ ID NO: 2); c) a peptide comprising the amino acid sequence STALPLWSNYSYDSA (SEQ ID NO: 3); d) a peptide comprising the amino acid sequence RTFGAKPPNIPFPRR (SEQ ID NO: 4); e) a peptide comprising the amino acid sequence KAIAFQSSPGSPVLL (SEQ ID NO: 5); f) a peptide comprising the amino acid sequence TCNLSDYTLPRARVL (SEQ ID NO: 6); g) a peptide comprising the amino acid sequence FLCPSPYPKVQPGL (SEQ ID NO: 7); h) a peptide comprising the amino acid sequence VPESIHKASLVCYRF (SEQ ID NO: 8); i) a peptide comprising the amino acid sequence SRPIARFYTWSQNTT (SEQ ID NO: 9); j) a peptide comprising the amino acid sequence FSPFRISELVYTLHP (SEQ ID NO: 10); k) a peptide comprising the amino acid sequence LPFNLAKPELYIFVQ (SEQ ID NO: 11); 1) a peptide comprising the amino acid sequence GRFLIRVTSSPLGPD (SEQ ID NO: 12; m) a peptide comprising the amino acid sequence TGSGLYLHQMVYLYQ (SEQ ID NO: 13); n) a peptide comprising the amino acid sequence FLIDSPLASIGPTSM (SEQ ID NO: 14); o) a peptide comprising the amino acid sequence FMIDSPLASIGPTSM (SEQ ID NO: 15); p) a peptide comprising the amino acid sequence FLSDPPAPPTSPGVV (SEQ ID NO: 16); q) a peptide comprising the amino acid sequence VLAWFSPLTLESSRL (SEQ ID NO: 17); r) a peptide comprising the amino acid sequence VIDLSGTRKSSSGTM (SEQ ID NO: 18); s) a peptide comprising the amino acid sequence VRKTTSHPPSYALLH (SEQ ID NO: 19); t) a peptide comprising the amino acid sequence TPPYRAALATPVLLL (SEQ ID NO: 20); u) a peptide comprising the amino acid sequence LSAPSPMFLPPVNPH (SEQ ID NO: 21); v) a peptide comprising the amino acid sequence HRPVKTPANAPTTMM (SEQ ID NO: 22); w) a peptide comprising the amino acid sequence CFNDPLDIVPPMLLL (SEQ ID NO: 23); x) a peptide comprising the amino acid sequence AHAPCSLFFPLSLRP (SEQ ID NO: 24); y) a peptide comprising the amino acid sequence KALYALHVPSMQVFA (SEQ ID NO: 25); z) a peptide comprising the amino acid sequence RPSRWPWQEPLPISI (SEQ ID NO: 26); and aa) a peptide comprising the amino acid sequence KVQIIPKDTLAPLPP (SEQ ID NO: 27) or fragments thereof which are capable of inhibiting growth of or killing Campylobacter spp.

In one embodiment, the method of inhibiting growth of or killing Campylobacter spp. comprises contacting the Campylobacter spp, with one or more peptides selected from the group consisting of a) a peptide comprising the amino acid sequence FSPFRISELVYTLHP (SEQ ID NO: 10); b) a peptide comprising the amino acid sequence GRFLIRVTSSPLGPD (SEQ ID NO: 12); c) a peptide comprising the amino acid sequence TGSGLYLHQMVYLYQ (SEQ ID NO: 13); d) a peptide comprising the amino acid sequence FLIDSPLASIGPTSM (SEQ ID NO: 14) or e) a peptide comprising the amino acid sequence FLSDPPAPPTSPGVV (SEQ ID NO: 16) or fragments of the one or more peptides which are capable of inhibiting growth of or killing Campylobacter spp.

In another embodiment, the method of inhibiting growth of or killing Campylobacter spp. comprises contacting the Campylobacter spp. with one or more peptides selected from the group consisting of a) a peptide comprising the amino acid sequence GRFLIRVTSSPLGPD (SEQ ID NO: 12; b) a peptide comprising the amino acid sequence TGSGLYLHQMVYLYQ (SEQ ID NO: 13) or c) a peptide comprising the amino acid sequence FLIDSPLASIGPTSM (SEQ ID NO: 14).

The methods of inhibiting growth of or killing Campylobacter spp. may comprise contacting the Campylobacter spp. with only one of abovementioned peptides or more than one of the abovementioned peptides as a combination.

In one embodiment, the one or more peptides bind to the Campylobacter spp. to inhibit growth of or kill the Campylobacter spp. The one or more peptides may bind preferentially to Campylobacter spp. to inhibit growth. The one or more peptides may bind specifically to Campylobacter spp. to inhibit growth.

The one or more peptides may be bactericidal. The one or more peptides may be bacteriostatic. The growth of Campylobacter spp may be inhibited by preventing growth of the Campylobacter spp. The growth of Campylobacter spp. may be inhibited by reducing or decreasing the growth rate of Campylobacter spp.

Growth of the Campylobacter spp. is inhibited by contacting the Campylobacter spp. with the one or more peptides. The Campylobacter spp. may be contacted with the peptide in any manner which inhibits the growth of Campylobacter spp. In one embodiment, the Campylobacter spp. is contacted with the peptide by incubating a culture of Campylobacter spp. with the one or more peptides.

The Campylobacter spp. may be killed by contacting the Campylobacter spp. with the one or more peptides. The Campylobacter spp. may be contacted with the peptide in any manner which kills the Campylobacter spp. In one embodiment, the Campylobacter spp. is contacted with the peptide by incubating a culture of Campylobacter spp. with the one or more peptides.

In one embodiment, the method of inhibiting growth may comprise inhibiting growth of or killing the Campylobacter spp. in a solution. In another embodiment, the method of inhibiting growth may comprise inhibiting growth of or killing the Campylobacter spp. on a surface.

The species of Campylobacter may be any species of Campylobacter in which the growth rate of the Campylobacter is reduced in the presence of the peptide relative to the growth rate of the Campylobacter in the absence of the peptide or which is killed by the peptide. The methods for determining the ability of a peptide to inhibit growth of or kill Campylobacter spp are described herein and are well within the capabilities of the person skilled in the art.

Campylobacter spp includes C. jejuni, C. coli, C. lari, C. Upsaliensis, C. concisus, C. curvus, C. fetus, C. graciius, C. helveticus, C. hyoilei, C. hyointestinalis, C. lanienae, C. mucosalis, C. rectus, C. showae, and C. sputorum.

### 3. Methods of Treating or Preventing Campylobacter Infection

As described above, the inventors have found that peptides described herein are capable of inhibiting growth of or killing Campylobacter spp. The inventors have also found that some peptides described herein show similarity to claudin 1 from animals such as Homo sapiens (Accession No. AAD22962); Rabbit (Oryctolagus cuniculus; Accession No. NP_{—}001082785); Rhesus monkey (Macaca mulatta; Accession No. XP_{—}001093143) and a flagella hook length control protein (flgE) from C. jejuni [Accession No's YP_{—}001001371; EAQ71955; NP_{—}281265; AAP34261) as described by: Hendrixson & DiRita. 2003. The inventors have also found that when some peptides described herein are contacted with C. jejuni, there is an effect on the morphology of the C. jejuni. Without wishing to be bound by theory, the inventors envisage that such peptides may inhibit the colonisation and invasion of the gastrointestinal cells of Campylobacter spp. The inventors therefore envisage that the peptides described herein can be used in the treatment or prevention of Campylobacter spp infections.

Accordingly, the invention further relates to a method of treating or preventing a Campylobacter spp infection in a subject comprising administering to the subject one or more peptides selected from the group consisting of: a) a peptide comprising the amino acid sequence EGVLGAALSAFSFDS (SEQ ID NO: 1) or a fragment thereof which is capable of treating or preventing Campylobacter spp infection; b) a peptide comprising the amino acid sequence QAQPCPNQPDGSVYA (SEQ ID NO: 2) or a fragment thereof which is capable of treating or preventing Campylobacter spp infection; c) a peptide comprising the amino acid sequence STALPLWSNYSYDSA (SEQ ID NO: 3) or a fragment thereof which is capable of treating or preventing Campylobacter spp infection; d) a peptide comprising the amino acid sequence RTFGAKPPNIPFPRR (SEQ ID NO: 4) or a fragment thereof which is capable of treating or preventing Campylobacter spp infection; e) a peptide comprising the amino acid sequence KAIAFQSSPGSPVLL (SEQ ID NO: 5) or a fragment thereof which is capable of treating or preventing Campylobacter spp infection; f) a peptide comprising the amino acid sequence TCNLSDYTLPRARVL (SEQ ID NO: 6) or a fragment thereof which is capable of treating or preventing Campylobacter spp infection; g) a peptide comprising the amino acid sequence NFLCPSPYPKVQPGL (SEQ ID NO: 7) or a fragment thereof which is capable of treating or preventing Campylobacter spp infection; h) a peptide comprising the amino acid sequence VPESIHKASLVCYRF (SEQ ID NO: 8) or a fragment thereof which is capable of treating or preventing Campylobacter spp infection; i) a peptide comprising the amino acid sequence SRPIARFYTWSQNTT (SEQ ID NO: 9) or a fragment thereof which is capable of treating or preventing Campylobacter spp infection; j) a peptide comprising the amino acid sequence FSPFRISELVYTLHP (SEQ ID NO: 10) or a fragment thereof which is capable treating or preventing Campylobacter spp infection; k) a peptide comprising the amino acid sequence LPFNLAKPELYIFVQ (SEQ ID NO: 11) or a fragment thereof which is capable of treating or preventing Campylobacter spp infection; 1) a peptide comprising the amino acid sequence GRFLIRVTSSPLGPD (SEQ ID NO: 12) or a fragment thereof which is capable of treating or preventing Campylobacter spp infection; m) a peptide comprising the amino acid sequence TGSGLYLHQMVYLYQ (SEQ ID NO: 13) or a fragment thereof which is capable of treating or preventing Campylobacter spp infection; n) a peptide comprising the amino acid sequence FLIDSPLASIGPTSM (SEQ ID NO: 14) or a fragment thereof which is capable of treating or preventing Campylobacter spp infection, o) a peptide comprising the amino acid sequence FMIDSPLASIGPTSM (SEQ ID NO: 15) or a fragment thereof which is capable of treating or preventing Campylobacter spp infection; p) a peptide comprising the amino acid sequence FLSDPPAPPTSPGVV (SEQ ID NO: 16) or a fragment thereof which is capable of treating or preventing Campylobacter spp; q) a peptide comprising the amino acid sequence VLAWFSPLTLESSRL (SEQ ID NO: 17) or a fragment thereof which is capable of treating or preventing treating or preventing Campylobacter spp; r) a peptide comprising the amino acid sequence VIDLSGTRKSSSGTM (SEQ ID NO: 18) or a fragment thereof which is capable of treating or preventing Campylobacter spp; s) a peptide comprising the amino acid sequence VRKTTSHPPSYALLH (SEQ ID NO: 19) or a fragment thereof which is capable of treating or preventing Campylobacter spp; t) a peptide comprising the amino acid sequence TPPYRAALATPVLLL (SEQ ID NO: 20) or a fragment thereof which is capable of treating or preventing Campylobacter spp; u) a peptide comprising the amino acid sequence LSAPSPMFLPPVNPH (SEQ ID NO: 21) or a fragment thereof which is capable of treating or preventing Campylobacter spp; v) a peptide comprising the amino acid sequence HRPVKTPANAPTTMM (SEQ ID NO: 22) or a fragment thereof which is capable of treating or preventing Campylobacter spp; w) a peptide comprising the amino acid sequence CFNDPLDIVPPMLLL (SEQ ID NO: 23) or a fragment thereof which is capable of treating or preventing Campylobacter spp; x) a peptide comprising the amino acid sequence AHAPCSLFFPLSLRP (SEQ ID NO: 24) or a fragment thereof which is capable of treating or preventing Campylobacter spp; y) a peptide comprising the amino acid sequence KALYALHVPSMQVFA (SEQ ID NO: 25) or a fragment thereof which is capable of treating or preventing Campylobacter spp; z) a peptide comprising the amino acid sequence RPSRWPWQEPLPISI (SEQ ID NO: 26) or a fragment thereof which is capable of treating or preventing Campylobacter spp; and aa) a peptide comprising the amino acid sequence KVQIIPKDTLAPLPP (SEQ ID NO: 27) or a fragment thereof which is capable of treating or preventing Campylobacter spp.

In one embodiment, the method of treating or preventing a Campylobacter spp infection in a subject comprises administering to the subject one or more peptides selected from the group consisting of: a) a peptide comprising the amino acid sequence FSPFRISELVYTLHP (SEQ ID NO: 10); b) a peptide comprising the amino acid sequence GRFLIRVTSSPLGPD (SEQ ID NO: 12; c) a peptide comprising the amino acid sequence TGSGLYLHQMVYLYQ (SEQ ID NO: 13); d) a peptide comprising the amino acid sequence FLIDSPLASIGPTSM (SEQ ID NO: 14) or e) a peptide comprising the amino acid sequence FLSDPPAPPTSPGVV (SEQ ID NO: 16)

In another embodiment, the method of treating or preventing a Campylobacter spp infection in a subject comprises administering to the subject one or more peptides selected from the group consisting of: a) a peptide comprising the amino acid sequence GRFLIRVTSSPLGPD (SEQ ID NO: 12; b) a peptide comprising the amino acid sequence TGSGLYLHQMVYLYQ (SEQ ID NO: 13) or c) a peptide comprising the amino acid sequence FLIDSPLASIGPTSM (SEQ ID NO: 14).

The term “subject” as used herein refers to any animal having a disease or condition which requires treatment. The subject may be a human, or may be a domestic or companion animal. While it is particularly contemplated that the peptides of the invention are suitable for use in the treatment of livestock species, such as poultry including chickens, ducks, geese and turkeys, it is also envisaged that such peptides will be suitable for the treatment of humans and companion animals such as dogs and cats, and other domestic animals such as horses, cattle and sheep, or zoo animals such as non-human primates, felids, canids, bovids, and ungulates.

Generally, the term “treating”, “treatment” and the like are used herein to mean affecting a subject, tissue or cell to obtain a desired pharmacological and/or physiological effect. “Treating” as used herein covers any treatment of infection in a vertebrate and includes: inhibiting the infection, i.e. arresting its development; or relieving or ameliorating the effects of the infection, ie. cause regression of the effects of the infection. “Preventing” is used herein to mean preventing the infection from occurring in a subject who may be predisposed to the infection, but has not yet been diagnosed as having the infection

The methods of this invention may involve the administration of one or more peptides in an amount sufficient to prevent or treat Campylobacter infection.

As used herein, the term “sufficient amount” means an amount of the one or more peptides of the present invention effective to yield a desired response, for example to prevent or treat an infection in subject which is susceptible to infection by administration of the peptides of the invention.

The specific “sufficient amount” will of course vary with such factors as the particular infection being treated, the physical condition and clinical history of the subject, the type of subject being treated, the duration of the treatment, the nature of concurrent therapy (if any), and the specific formulations employed and the structure of the peptides or fragments or variants thereof.

### 4. Compositions

The peptides of the invention may be contacted with the Campylobacter spp. in the form of a composition. The peptides of the invention may also be administered to a subject as a pharmaceutical composition comprising one or more peptides of the invention and a pharmaceutically acceptable carrier.

As used herein, a “pharmaceutical acceptable carrier” is a pharmaceutically acceptable solvent, suspending agent, excipient or vehicle for delivering peptides to a subject. The carrier may be liquid or solid, and is selected with the planned manner of administration in mind.

Pharmaceutically acceptable carriers include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol and polyhydric alcohols, non-toxic excipients, including salts, preservatives, buffers and the like, as described, for instance, in Remington's Pharmaceutical Sciences, 20th ed. Williams & Wilkins (2000) and The British National Formulary 43rd ed. (British Medical Association and Royal Pharmaceutical Society of Great Britain, 2002; http://bnf.rhn.net). The pH and exact concentration of the various components of the pharmaceutical composition are adjusted according to routine skills in the art. See Goodman and Gilman's The Pharmacological Basis for Therapeutics (7th ed., 1985).

The specific dose level of the peptides or compositions of the invention for any particular subject will depend upon a variety of factors including the activity of the specific peptide employed, the age, body weight, general health, sex, diet, time of administration, route of administration, rate of excretion, drug combination and the severity of the particular infection.

For in vitro use the peptides may be added or dissolved in an appropriate biologically acceptable buffer and added to a cell or tissue. Biologically acceptable buffers include TPS, saline and PBS.

The peptides of the invention may be used as a component of or in developing vaccines for Campylobacter spp infection in humans or poultry or other animals. There are several routes available for delivering vaccines as would be understood by the person skilled in the art. In one embodiment peptides of the invention are used to identify a bacterial receptor for use in a sub-unit vaccine. In this embodiment a receptor to which one or more of the peptides binds is identified by bringing Campylobacter spp into contact with one or more said peptides, allowing binding to take place and identifying the receptor. The peptide of the invention may be immobilised to a solid support such as a filter, beads, a column, dipstick or gel, or may be part of a fusion protein or part of the phage coat. The receptor so-identified is isolated and/or synthesised in a manner known to the person skilled in the art and then used as the antigen component of a sub-unit vaccine. Preparation of the vaccine and suitable vehicles and components such as adjuvants are well known to the person skilled in the art. In another embodiment the peptides of the invention may be used as components of a vaccine such as in a peptide-based vaccine, which may utilise one or more phage-displayed peptides of the invention. Equally an attenuated bacterial strain may display the peptide and in this regard Wyszynska et al., 2004 describes the oral administration of chickens with an avirulent Salmonella accine strain carrying Campylobacter antigens.

Any convenient method of delivering the polypeptides to a subject is envisaged. In one embodiment one or more peptides of the invention is expressed in a vector in plants (an example is provided by Fang et al. 2006). The transgenic plant (e.g. grain etc) expressing the therapeutic peptide is fed to poultry. In another embodiment the delivery vehicle is as described by Lazarev et al. 2004. In this embodiment a recombinant plasmid vector is prepared and administered to chickens as an aerosol.

It will be apparent to the person skilled in the art that while the invention has been described in some detail for the purposes of clarity and understanding, various modifications and alterations to the embodiments and methods described herein may be made without departing from the scope of the inventive concept disclosed in this specification.

### 5. Methods of Diagnosis

As described above, the inventors have found the peptides described herein are capable of diagnosing Campylobacter spp infection. Accordingly the present invention relates to a method of diagnosing a Campylobacter spp. infection in a subject comprising:

1) providing a sample from the subject;
2) contacting the sample with one or more peptides under conditions which permit binding of the one or more peptides to Campylobacter spp.; and
3) determining if the one or more peptides bind to a Campylobacter spp. in the sample, wherein the one or more peptides are selected from the group consisting of:
a) a peptide comprising the amino acid sequence EGVLGAALSAFSFDS (SEQ ID NO: 1) or a fragment thereof which is capable of diagnosing Campylobacter spp infection; b) a peptide comprising the amino acid sequence QAQPCPNQPDGSVYA (SEQ ID NO: 2) or a fragment thereof which is capable of diagnosing Campylobacter spp infection;
c) a peptide comprising the amino acid sequence STALPLWSNYSYDSA (SEQ ID NO: 3) or a fragment thereof which is capable of diagnosing Campylobacter spp infection; d) a peptide comprising the amino acid sequence RTFGAKPPNIPFPRR (SEQ ID NO: 4) or a fragment thereof which is capable of diagnosing Campylobacter spp infection;
e) a peptide comprising the amino acid sequence KAIAFQSSPGSPVLL (SEQ ID NO: 5) or a fragment thereof which is capable of diagnosing Campylobacter spp infection;
f) a peptide comprising the amino acid sequence TCNLSDYTLPRARVL (SEQ ID NO: 6) or a fragment thereof which is capable of diagnosing Campylobacter spp infection; g) a peptide comprising the amino acid sequence NFLCPSPYPKVQPGL (SEQ ID NO: 7) or a fragment thereof which is capable of diagnosing Campylobacter spp infection; h) a peptide comprising the amino acid sequence VPESIHKASLVCYRF (SEQ ID NO: 8) or a fragment thereof which is capable of diagnosing Campylobacter spp infection;
i) a peptide comprising the amino acid sequence SRPIARFYTWSQNTT (SEQ ID NO: 9) or a fragment thereof which is capable of diagnosing Campylobacter spp infection;
j) a peptide comprising the amino acid sequence FSPFRISELVYTLHP (SEQ ID NO: 10) or a fragment thereof which is capable diagnosing Campylobacter spp infection;
k) a peptide comprising the amino acid sequence LPFNLAKPELYIFVQ (SEQ ID NO: 11) or a fragment thereof which is capable of diagnosing Campylobacter spp infection;
l) a peptide comprising the amino acid sequence GRFLIRVTSSPLGPD (SEQ ID NO: 12) or a fragment thereof which is capable of diagnosing Campylobacter spp infection;
m) a peptide comprising the amino acid sequence TGSGLYLHQMVYLYQ (SEQ ID NO: 13) or a fragment thereof which is capable of diagnosing Campylobacter spp infection;
n) a peptide comprising the amino acid sequence FLIDSPLASIGPTSM (SEQ ID NO: 14) or a fragment thereof which is capable of diagnosing Campylobacter spp infection;
o) a peptide comprising the amino acid sequence FMIDSPLASIGPTSM (SEQ ID NO: 15) or a fragment thereof which is capable of diagnosing Campylobacter spp infection;
p) a peptide comprising the amino acid sequence FLSDPPAPPTSPGVV (SEQ ID NO: 16) or a fragment thereof which is capable of diagnosing Campylobacter spp infection;
q) a peptide comprising the amino acid sequence VLAWFSPLTLESSRL (SEQ ID NO: 17) or a fragment thereof which is capable of diagnosing Campylobacter spp infection;
r) a peptide comprising the amino acid sequence VIDLSGTRKSSSGTM (SEQ ID NO: 18) or a fragment thereof which is capable of diagnosing Campylobacter spp infection;
s) a peptide comprising the amino acid sequence VRKTTSHPPSYALLH (SEQ ID NO: 19) or a fragment thereof which is capable of diagnosing Campylobacter spp infection;
t) a peptide comprising the amino acid sequence TPPYRAALATPVLLL (SEQ ID NO: 20) or a fragment thereof which is capable of diagnosing Campylobacter spp infection;
u) a peptide comprising the amino acid sequence LSAPSPMFLPPVNPH (SEQ ID NO: 21) or a fragment thereof which is capable of diagnosing Campylobacter spp infection;
v) a peptide comprising the amino acid sequence HRPVKTPANAPTTMM (SEQ ID NO: 22) or a fragment thereof which is capable of diagnosing Campylobacter spp infection;
w) a peptide comprising the amino acid sequence CFNDPLDIVPPMLLL (SEQ ID NO: 23) or a fragment thereof which is capable of diagnosing Campylobacter spp infection;
x) a peptide comprising the amino acid sequence AHAPCSLFFPLSLRP (SEQ ID NO: 24) or a fragment thereof which is capable of diagnosing Campylobacter spp infection;
y) a peptide comprising the amino acid sequence KALYALHVPSMQVFA (SEQ ID NO: 25) or a fragment thereof which is capable of diagnosing Campylobacter spp infection;
z) a peptide comprising the amino acid sequence RPSRWPWQEPLPISI (SEQ ID NO: 26) or a fragment thereof which is capable of diagnosing Campylobacter spp infection; and
   aa) a peptide comprising the amino acid sequence KVQIIPKDTLAPLPP (SEQ ID NO: 27) or a fragment thereof which is capable of diagnosing Campylobacter spp infection.

In one embodiment the method employs a peptide comprising the amino acid sequence LPFNLAKPELYIFVQ (SEQ ID NO: 11).

In one embodiment the peptides may be used for diagnosis by way of detection of binding to bacteria of Campylobacter spp. More specifically, the method of “detecting” binding partner may comprise contacting a sample putatively containing bacteria of Campylobacter spp. with peptides which have been detectably labelled. In an embodiment the peptide which binds Campylobacter spp. may be one of a plurality of capture agents immobilized on an array. For example, a peptide can be detectably labelled with biotin and then contacted with the sample, thereby allowing any bacteria of Campylobacter spp. to be bound by the labelled peptides. The presence of the bacteria of interest at a particular location, such as that corresponding to a particular peptide serving as one of the capture agents in the array, can be determined by addition of streptavidin or avidin, which is then detected by a streptavidin or avidin specific antibody conjugated to a detectable label. Other types of detectable labelling of the peptides can also be employed, including, but not limited to, fluorescent Cy3 and Cy5 labels, gold particles, and radiolabels.

As an alternative to biotin, the peptides may be labelled by chemiluminescence, enhanced chemiluminescence, or a radiolabel. Alternatively, a secondary antibody may be labelled with, for example, horseradish peroxidase or another detectable enzyme or label allowing colorimetric detection of the peptide complex. Other methods of labeling and detecting proteins are known to persons skilled in the art.

In an embodiment the peptides of the invention may be incorporated into a phage display library. The phage display library may be used for high-throughput screening of protein interactions and so detect the presence of a receptor for the peptide on the surface of Campylobacter spp. Methods for introducing a peptide to a phage display library are well understood by the person skilled in the art and described, for example, by Smith & Petrenko (1997). Briefly, the methods involve ligation of a target DNA fragment into genes encoding the coat of a phage such as into the pIII or pVIII of a filamentous phage. The filamentous phage strains M13, fd and f1 are commonly used as vectors. The introduced DNA fragment is expressed on the surface of the phage particle as part of the coat protein. If several DNA fragments are incorporated into the gene then this becomes a library of those DNA fragments. The phage gene and inserted DNA is then transformed into bacterial cells such as male E. coli bacterial cells, in particular TG1 E. coli cells. The phage particles will not be released upon maturation of the bacterial cells until they are infected with a helper phage but then will display the relevant protein fragment as part of their outer coats. An assay may also be based upon the possibility that only a sample containing Campylobacter spp. can serve as a suitable host for the phage and therefore that amplification will only take place when a bacterium of Campylobacter spp. is present in the sample. For example, Biotec Laboratories (Ipswich, UK) owns Phage Amplification Technology which has the potential for application to a number of different bacteria using the presence or absence of phage amplification for the diagnosis of infection. In this system a phage infects the bacteria in question and a potent viricide destroys all phages that have not infected a bacterium. The phage continues replication in the viable bacterial cells and are released by cell lysis to reinfect non-pathogenic sensor cells. Zones of clearing (plaques) on a lawn of sensor cells signify a positive result in such a test.

### EXAMPLES

The invention will now be described in detail by way of reference only to the following non-limiting examples and drawings.

Growth of Campylobacter jejuni

Poultry isolates of C. jejuni were provided by Dr. Pat Blackall (QDPI, Australia) and C. jejuni ACM 3393 (NCTC11351) was purchased from the Australian Collection of Microorganisms (Queensland, Australia). C. jejuni was grown on either pre-purchased Campylobacter agar plates (Oxoid, Australia) or blood agar plates [40 g/L Blood Agar Base #2 (Oxoid), 5% (v/v) defibrinated horse blood (Oxoid), and Campylobacter Growth Supplement (0.25 g/L sodium pyruvate, 0.25 g/L sodium betabisulphite and 0.25 g/L ferrous sulphate; Oxoid)]. A microaerophilic atmosphere was provided by CampyGen Sachets (Oxoid) in 2.5 L anaerobe jars or alternatively by GasPak E2 Campy Pouches (BD Biosciences). Cultures were grown for 1-2 d at 37° C.

For growth in liquid media, C. jejuni was resuspended in Nutrient Broth No. 2 (consists of 10 g/L Lab-Lemco powder, 10 g/L peptone and 5 g/L sodium chloride; Oxoid) supplemented with Campylobacter Growth Supplement (0.25 g/L sodium pyruvate, 0.25 g/L sodium betabisulphite and 0.25 g/L ferrous sulphate; Oxoid). A microaerophilic environment was provided by GasPak E2 Campy Pouches (BD Biosciences) and cultures were grown for 1-2 d at 37° C. and 180 rpm.

### Affinity Selection

The f88-4/15-mer peptide phage-display library (provided by Dr. G. P. Smith, University of Missouri-Columbia, USA) was used to affinity select for phage peptides binding to the outer surface of C. jejuni. This library consists of filamentous fd-tet phage displaying random 15-mer peptides fused to 150-300 copies of major coat protein pVIII (Scott & Smith, 1990) and contained a complexity of 2×10⁹ peptidevariants/mL.

FIG. 1 shows the selection schemes for isolating phage clones that bound to C. jejuni. In Selection Scheme A (FIG. 1), the phage-display library was directly selected against C. jejuni strain ACM 3393, resulting in Pool A phage. In brief: 10¹° transducing units (TU; Smith & Scott, 1993) of the f88-4/15-mer phage library was incubated with 10⁹ CFU of C. jejuni (strain ACM 3393) resuspended in a final volume of 3 mL PBSg (phosphate buffered saline pH 7.0 with 0.1% gelatin). This C. jejuni-phage solution was kept in suspension by agitation on an orbital shaker at room temperature in a petri dish. After 20 min, phage binding to C. jejuni was separated from non-binding phage in solution by a series of washing and centrifugation steps (3,000 rpm for 2 min) using either 1 mL of PBSg (pH 7.0) or TBST (TBS pH 7.5 containing 0.5% Tween 20) as parallel experiments (PBSg washed or TBST washed in FIG. 1). The phage remaining bound to C. jejuni was eluted from the pelleted C. jejuni by the addition of elution buffer (0.1 N HCl, pH 2.2 with glycine; 1 g/L bovine serum albumin) for 10 min at room temperature and immediately neutralised with 1 M Tris-Cl (pH 9.0). The phage was amplified by infecting “starved” Escherichia coli K91BlueKan cells (Yu & Smith, 1996), twice purified by polyethylene glycol (PEG) precipitation and resuspended in TBS buffer (50 mM Tris-Cl pH 7.5, 150 mM NaCl) (Smith & Scott, 1993). This amplified phage was titered in E. coli K91BluKan cells prior to being re-applied to freshly prepared C. jejuni cells as above. Affinity selections against C. jejuni was continued for a total of three-affinity selections and two amplification steps.

In Selection Scheme B (FIG. 1), a depletion or negative step was performed against C. jejuni strain ACM 3393, prior to three rounds of affinity selection against C. jejuni strain ACM 3393. This resulted in Pool B phage (BT and BP phage; FIG. 1). In Selection Scheme C (FIG. 1), two depletion/negative steps were performed against C. jejuni strain ACM 3393, prior to three rounds of affinity selection against C. jejuni strain C338. This resulted in Pool C phage (CT and CP phage; FIG. 1). In Selection Scheme D (FIG. 1), three depletion/negative steps were performed (twice against C. jejuni strain ACM 3393 and once against strain C338), prior to three affinity selections against C. jejuni strain C338.

Phage eluted from the final (3^{rd}) round of each of the selection schemes were used to infect Escherichia coli K91BlueKan cells. These cells were plated on LB (Luria Broth) plates containing 40 mg/L tetracycline, to select for bacteria containing phage. Bacterial colonies each containing a single phage population were randomly selected and propagated for sequencing and testing in antimicrobial assays. Individual phage clones were randomly picked from each of the selection strategies.

### DNA Sequencing and Peptide Analysis

Single-stranded DNA was isolated from individual phage clones and sequenced from the 3′ end of the pVIII gene using an ABI Prism 377 automated sequencer at the DNA Analysis Facility (Macquarie University, NSW, Australia). The DNA sequences were conceptually translated into amino acids using the “translate” program on the proteomics server of the Swiss Institute of Bioinformatics Expert Protein Analysis System (ExPASy; http://www.expasy.ch). Homologies of the translated peptide sequences with known proteins were obtained using the BLAST program (NCBI; http://www.ncbi.nlm.gov/blast).

### Inhibition of Growth of a Campylobacter Species

Representative affinity-selected phage peptides were tested for their ability to inhibit the growth of C. jejuni strains in vitro. Each phage clone was amplified by E. coli infection and twice purified by PEG precipitation and resuspended in TBS buffer (pH 7.5). Phage concentrations were calculated as virions/4 using the ultraviolet (UV) absorbance measurements on a spectrophotometer (Smith & Scott, 1993). A version of the microdilution susceptibility test was used to determine the antimicrobial activity of each of the phage-displayed peptides. In brief: each of the C. jejuni strains was resuspended to an OD₅₉₅ of 0.2 in Nutrient Broth and then diluted to 10⁴-10⁵ CFU/mL in Nutrient Broth containing Campylobacter growth supplement. Each well in a microtiter plate was inoculated with 150 μL, of 1-5×10⁵ CFU/mL of bacteria and 45 μL of individual phage-displayed peptides in TBS (pH 7.5). Controls included TBS without phage-displayed peptides and TBS containing unselected phage libraries. A microaerophilic environment was provided by GasPak E2 Campy Pouches (BD Biosciences). After 24-48 h at 37° C. without shaking, the optical densities of the bacterial cultures were measured at OD₅₉₅ nm in a plate reader (Victor2, Perkin Elmer).

The number of viable bacteria in the 96 well plates was determined by performing colony counts on the diluted inoculum and plating onto blood agar plates. Colony counts on blood agar plates were also used to determine the initial inoculum of bacteria used in the assay. A Gram Stain Kit was used to check the morphology of C. jejuni after incubation with the phage peptides.

Overall, ten of the phage clones were found to inhibit the growth of C. jejuni in vitro, as depicted in Table 2. Single-stranded DNA was isolated from these 10 clones and peptide-encoding region on gene pVIII sequenced (as shown in Table 2). Two of the clones contained the same sequence, as represented by CP3/3 (FLIDSPLASIGPTSM in Table 2) and CP3/4 (FMIDSPLASIGPTSM in Table 2). The remaining six clones encoded unique peptide sequences (Table 2).

Using the microdilution susceptibility test, some of the phage clones were able to inhibit the growth of C. jejuni in vitro, as shown in Table 2. For example, BT3/4 inhibited the growth of C. jejuni in vitro at a bacterial concentration of 1×10⁴ CFU/mL. CP3/1 inhibited the growth of C. jejuni by 97% when the initial bacterial inoculum was 2.8×10⁴ CFU/mL and by 89% when the initial bacterial inoculum was 2.8×10⁴ CFU/mL. CP3/2 inhibited the growth of C. jejuni by 92% when the initial bacterial inoculum was 2.8×10⁴ CFU/mL and by 87% when the bacterial inoculum was 1.6×10⁵ CFU/mL. In addition, these phage peptides were able to affect the morphology of C. jejuni as depicted in FIG. 2.

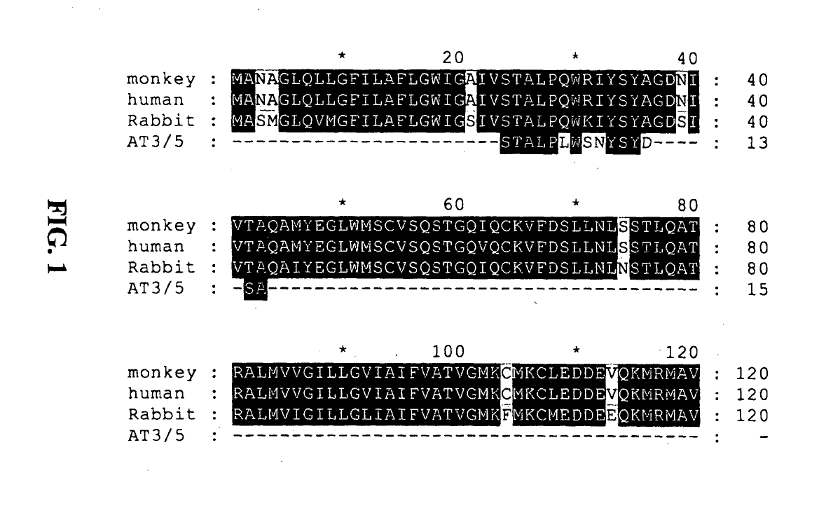

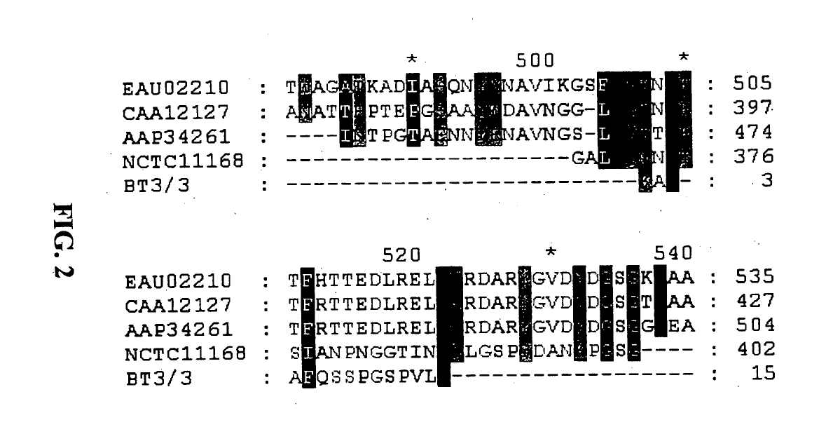

Sequence analysis using BLAST (National Centre of Biotechnology Information) indicate that some of the peptides showed similarities to known proteins in the database. For example AT3/5 from Table 1, showed similarity to claudin 1, an integral membrane protein, which is part of the tight junction proteins in the gastrointestinal tract of many animal species (FIG. 3). In a mouse model of gastrointestinal infection, Helicobacter pylori was found to disrupt the expression of claudin 1. BT3/3 in Table 1 showed similarity to a flagella hook length control protein (flgE) from C. jejuni (FIG. 4). This protein is required for C. jejuni motility and virulence in vivo. Together these results indicate that some of the phage peptides may act by disrupting the ability of Campylobacter to colonise and invade gastrointestinal cells.

It is to be understood that a reference herein to a prior art publication does not constitute an admission that the publication forms part of the common general knowledge in the art in Australia, or any other country.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

### REFERENCES

References cited herein are listed on the following pages, and are incorporated herein by this reference.
Altekruse, S. F.; Stern, N. J.; Fields, P. I., and Swerdlow, D. L. (1999). Campylobacter jejuni—an emerging foodborne pathogen. Emerg Infect Dis. 28; 5(1):28-35.
Berrang, M. E.; Buhr, R. J., and Cason, J. A. (2000) Campylobacter recovery from external and internal organs of commercial broiler carcass prior to scalding. Poult Sci. 79(2):286-90.
Bishop-Hurley S. L et al. 2005 Antimicrobial Agents & Chemotherapy 49 (7): 2972-2978).
Cason, J. A.; Bailey, J. S.; Stern, N. J.; Whittemore, A. D., and Cox, N. A. (1997) Relationship between aerobic bacteria, salmonellae and Campylobacter on broiler carcasses. Poult Sci. 76(7):1037-41.
Denis, M.; Refregier-Petton, J.; Laisney, M. J.; Ermel, G., and Salvat, G. (2001) Campylobacter contamination in French chicken production from farm to consumers. Use of a PCR assay for detection and identification of Campylobacter jejuni and Camp. coli. J Appl Microbiol. 91(2):255-67.
Dill, D.; Stanker, L. H.; Young, C. R. 1999. Detection of salmonella in poultry using a silicon chip-based biosensor. J. Biochem. Biophys. Methods 41: 61-67.
Evans, S. J. and Sayers, A. R. (2000) A longitudinal study of campylobacter infection of broiler flocks in Great Britain. Prey Vet Med. 46(3):209-23.
Fang, Z. D.; Laskey, J. G.; Huang, S.; Bilyeu, K. D.; Morris, R. O.; Schmidt, F. J.; English, J. T. (2006) Combinatorially selected defense peptides protect plant roots from pathogen infection. PNAS 103 (49): 18444-9.
Ge, B.; White, D. G.; McDermott, P. F.; Girard, W.; Zhao, S.; Hubert, S., and Meng, J. (2003) Antimicrobial-resistant Campylobacter species from retail raw meats. Appl Environ Microbiol. 69(5): 3005-7.
Gupta, A.; Nelson, J. M.; Barrett, T. J.; Tauxe, R. V.; Rossiter, S. P.; Friedman, C. R.; Joyce, K. W.; Smith, K. E.; Jones, T. F.; Hawkins, M. A.; Shiferaw, B.; Beebe, J. L.; Vugia, D. J.; Rabatsky-Ehr, T.; Benson, J. A.; Root, T. P., and Angulo, F. J. (2004) Antimicrobial resistance among Campylobacter strains, United States, 1997-2001. Emerg Infect Dis. 10(6): 1102-9.
Hendrixson, D. R.; DiRita, V. J. 2003. Transcription of sigma 54-dependent but not sigma 28-dependent flagellar genes in Campylobacter jejuni is associated with formation of the flagellar secretory apparatus. Mol. Microbiol. 50 (2): 687-702.
Lazarev, V. N.; Stipkovits, L.; Biro, J.; Miklodi, D.; Shkarupeta, M. M.; Titova, G. A.; Akopian, T. A.; Govorun, V. M. 2004. Induced expression of the antimicrobial peptide melittin inhibits experimental infection by Mycoplasma gallisepticum in chickens. Microbes and Infection 6: 536-541.
Morishita, T. Y.; Aye, P. P.; Harr, B. S.; Cobb, C. W., and Clifford, J. R. (1997) Evaluation of an avian-specific probiotic to reduce the colonization and shedding of Campylobacter jejuni in broilers. Avian Dis. 41(4):850-5.
Musgrove, M. T.; Cason, J. A.; Fletcher, D. L.; Stern, N. J.; Cox, N. A., and Bailey, J. S. (1997) Effect of cloacal plugging on microbial recovery from partially processed broilers. Poult Sci. 76(3):530-3.
Northcutt, J. K.; Smith, D. P.; Musgrove, M. T.; Ingram, K. D., and Hinton, A. Jr. (2005) Microbiological impact of spray washing broiler carcasses using different chlorine concentrations and water temperatures. Poult Sci. 84(10):1648-52.
Prats, G.; Mirelis, B.; Llovet, T.; Munoz, C.; Miro, E., and Navarro, F. (2000) Antibiotic resistance trends in enteropathogenic bacteria isolated in 1985-1987 and 1995-1998 in Barcelona. Antimicrob Agents Chemother. 44(5): 1140-5.
Rivoal, K.; Denis, M.; Salvat, G.; Colin, P., and Ermel, G. (1999) Molecular characterization of the diversity of Campylobacter spp. isolates collected from a poultry slaughterhouse: analysis of cross-contamination. Lett Appl Microbiol. 29(6):370-4.
Smith, G. P. and Petrenko, V. A., Phage Display Chem. Rev. 1997, 97, 391-410. Stern, N. J.; Svetoch, E. A.; Eruslanov, B. V.; Kovalev, Y. N.; Volodina, L. I.; Perelygin, V. V.; Mitsevich, E. V.; Mitsevich, I. P., and Levchuk, V. P. (2005) Paenibacillus polymyxa purified bacteriocin to control Campylobacter jejuni in chickens. J Food Prot. 68(7):1450-3.
Stewart,J. M., et al (1984) Solid Phase Synthesis (Pierce Chemical Co., 2^{nd} ed.)
Wallace, J. S.; Stanley, K. N., and Jones, K. (1998) The colonization of turkeys by thermophilic campylobacters. J Appl Microbiol. 85(2):224-30.
Wren, B. W.; Linton, D.; Dorrell, N., and Karlyshev, A. V. (2001) Post genome analysis of Campylobacter jejuni. Symp Ser Soc Appl Microbiol. (30):36S-44S.
Wyszynska, A.; Rackzko, A.; L is, M.; Jagusztyn-Krynicka, E. K. 2004. Oral immunisation of chickens with avirulent Salmonella vaccine strain carrying C. jejuni 72Dz/92 cjaA gene elicits specific humoral immune response associated with protection against challenge with wild-type Campylobacter. Vaccine 22: 1379-1389.
Zhao, C.; Ge, B.; De Villena, J.; Sudler, R.; Yeh, E.; Zhao, S.; White, D. G.; Wagner, D., and Meng, J. (2001) Prevalence of Campylobacter spp., Escherichia coli, and Salmonella serovars in retail chicken, turkey, pork, and beef from the Greater Washington, D.C., area. Appl Environ Microbiol. 67(12):5431-6.
Zwick, M. B.; Bonnycastle, L. L.; Noren, K. A.; Venturini, S.; Leong, E.; Barbas, C. F.; Noren, C. J.; Scott, J. K. 1998 The maltose-binding protein as a scaffold for monovalent display of peptides derived from phage libraries. Anal Biochem 264: 87-97.

TABLE 1
Amino acid sequences of the phage-displayed
peptides.

| Phage clones | Amino acid sequence | SEQ ID NO |
|---|---|---|
| AT3/3 | EGVLGAALSAFSFDS | 1 |
| AT3/4 | QAQPCPNQPDGSVYA | 2 |
| AT3/5 | STALPLWSNYSYDSA | 3 |
| AP3/5 | RTFGAKPPNIPFPRR | 4 |
| BT3/3 | KAIAFQSSPGSPVLL | 5 |
| BT3/4 | TCNLSDYTLPRARVL | 6 |
| BT3/7 | NFLCPSPYPKVQPGL | 7 |
| BP3/5 | VPESIHKASLVCYRF | 8 |
| BP3/9 | SRPIARFYTWSQNTT | 9 |
| CT3/2 | FSPFRISELVYTLHP | 10 |
| CT3/4 | LPFNLAKPELYIFVQ | 11 |
| CP3/1 | GRFLIRVTSSPLGPD | 12 |
| CP3/2 | TGSGLYLHQMVYLYQ | 13 |
| CP3/3 | FLIDSPLASIGPTSM | 14 |
| CP3/4 | FMIDSPLASIGPTSM | 15 |
| CP3/5 | FLIDSPLASIGPTSM | 14 |
| CP3/6 | FMIDSPLASIGPTSM | 15 |
| DP3/5 | FLSDPPAPPTSPGVV | 16 |
| CP2/5 | VLAWFSPLTLESSRL | 17 |
| CP3/8 | VIDLSGTRKSSSGTM | 18 |
| CP3/9 | VRKTTSHPPSYALLH | 19 |
| CP3/12 | TPPYRAALATPVLLL | 20 |
| CT3/6 | LSAPSPMFLPPVNPH | 21 |
| CT3/7 | HRPVKTPANAPTTMM | 22 |
| CT3/9 | CFNDPLDIVPPMLLL | 23 |
| AT4/3 | AHAPCSLFFPLSLRP | 24 |
| AT4/5 | KALYALHVPSMQVFA | 25 |
| AT4/8 | RPSRWPWQEPLPISI | 26 |
| AT4/2 | KVQIIPKDTLAPLPP | 27 |

TABLE 2
Amino acid sequences of the phage-displayed
peptides and their antimicrobial activities
against C. jejuni strain C338

| | | % inhibition |
|---|---|---|
| Phage clones | Amino acid sequences | of growth |
| BT3/4 | TCNLSDYTLPRARVL | 60% |
| CP3/1 | GRFLIRVTSSPLGPD | 96.7% |
| CP3/2 | TGSGLYLHQMVYLYQ | 92% |
| CP3/3 | FLIDSPLASIGPTSM | 99.3% |

1. An isolated peptide which binds to Campylobacter spp., wherein the peptide is selected from the group consisting of:
a) a peptide comprising the amino acid sequence EGVLGAALSAFSFDS (SEQ ID NO: 1) or a fragment thereof which is capable of binding to Campylobacter spp;
b) a peptide comprising the amino acid sequence QAQPCPNQPDGSVYA (SEQ ID NO: 2) or a fragment thereof which is capable of binding to Campylobacter spp;
c) a peptide comprising the amino acid sequence STALPLWSNYSYDSA (SEQ ID NO: 3) or a fragment thereof which is capable of binding to Campylobacter spp;
d) a peptide comprising the amino acid sequence RTFGAKPPNIPFPRR (SEQ ID NO: 4) or a fragment thereof which is capable of binding to Campylobacter spp;
e) a peptide comprising the amino acid sequence KAIAFQSSPGSPVLL (SEQ ID NO: 5) or a fragment thereof which is capable of binding to Campylobacter spp;
f) a peptide comprising the amino acid sequence TCNLSDYTLPRARVL (SEQ ID NO: 6) or a fragment thereof which is capable of binding to Campylobacter spp;
g) a peptide comprising the amino acid sequence NFLCPSPYPKVQPGL (SEQ ID NO: 7) or a fragment thereof which is capable of binding to Campylobacter spp;
h) a peptide comprising the amino acid sequence VPESIHKASLVCYRF (SEQ ID NO: 8) or a fragment thereof which is capable of binding to Campylobacter spp;
i) a peptide comprising the amino acid sequence SRPIARFYTWSQNTT (SEQ ID NO: 9) or a fragment thereof which is capable of binding to Campylobacter spp;
j) a peptide comprising the amino acid sequence FSPFRISELVYTLHP (SEQ ID NO: 10) or a fragment thereof which is capable of binding to Campylobacter spp;
k) a peptide comprising the amino acid sequence LPFNLAKPELYIFVQ (SEQ ID NO: 11) or a fragment thereof which is capable of binding to Campylobacter spp; 1) a peptide comprising the amino acid sequence GRFLIRVTSSPLGPD (SEQ ID NO: 12) or a fragment thereof which is capable of binding to Campylobacter spp;
m) a peptide comprising the amino acid sequence TGSGLYLHQMVYLYQ (SEQ ID NO: 13) or a fragment thereof which is capable of binding to Campylobacter spp;
n) a peptide comprising the amino acid sequence FLIDSPLASIGPTSM (SEQ ID NO: 14) or a fragment thereof which is capable of binding to Campylobacter spp
o) a peptide comprising the amino acid sequence FMIDSPLASIGPTSM (SEQ ID NO: 15) or a fragment thereof which is capable of binding to Campylobacter spp;
p) a peptide comprising the amino acid sequence FLSDPPAPPTSPGVV (SEQ ID NO: 16) or a fragment thereof which is capable of binding to Campylobacter spp;
q) a peptide comprising the amino acid sequence VLAWFSPLTLESSRL (SEQ ID NO: 17) or a fragment thereof which is capable of binding to Campylobacter spp;
r) a peptide comprising the amino acid sequence VIDLSGTRKSSSGTM (SEQ ID NO: 18) or a fragment thereof which is capable of binding to Campylobacter spp;
s) a peptide comprising the amino acid sequence VRKTTSHPPSYALLH (SEQ ID NO: 19) or a fragment thereof which is capable of binding to Campylobacter spp;
t) a peptide comprising the amino acid sequence TPPYRAALATPVLLL (SEQ ID NO: 20) or a fragment thereof which is capable of binding to Campylobacter spp;
u) a peptide comprising the amino acid sequence LSAPSPMFLPPVNPH (SEQ ID NO: 21) or a fragment thereof which is capable of binding to Campylobacter spp;
v) a peptide comprising the amino acid sequence HRPVKTPANAPTTMM (SEQ ID NO: 22) or a fragment thereof which is capable of binding to Campylobacter spp;
w) a peptide comprising the amino acid sequence CFNDPLDIVPPMLLL (SEQ ID NO: 23) or a fragment thereof which is capable of binding to Campylobacter spp;
x) a peptide comprising the amino acid sequence AHAPCSLFFPLSLRP (SEQ ID NO: 24) or a fragment thereof which is capable of binding to Campylobacter spp;
y) a peptide comprising the amino acid sequence KALYALHVPSMQVFA (SEQ ID NO: 25) or a fragment thereof which is capable of binding to Campylobacter spp;
z) a peptide comprising the amino acid sequence RPSRWPWQEPLPISI (SEQ ID NO: 26) or a fragment thereof which is capable of binding to Campylobacter spp; and
aa) a peptide comprising the amino acid sequence KVQIIPKDTLAPLPP (SEQ ID NO: 27) or a fragment thereof which is capable of binding to Campylobacter spp.

2. An isolated peptide according to claim 1, wherein the peptide binds specifically to Campylobacter spp.

3. A method of inhibiting growth of or killing a Campylobacter species comprising contacting the species with one or more peptides in an amount sufficient to inhibit growth of or kill the species, wherein the one or more peptides are selected from the group consisting of:
a) a peptide comprising the amino acid sequence EGVLGAALSAFSFDS (SEQ ID NO: 1) or a fragment thereof which is capable of inhibiting growth of or killing Campylobacter spp;
b) a peptide comprising the amino acid sequence QAQPCPNQPDGSVYA (SEQ ID NO: 2) or a fragment thereof which is capable of inhibiting growth of or killing Campylobacter spp;
c) a peptide comprising the amino acid sequence STALPLWSNYSYDSA (SEQ ID NO: 3) or a fragment thereof which is capable of inhibiting growth of or killing Campylobacter spp;
d) a peptide comprising the amino acid sequence RTFGAKPPNIPFPRR (SEQ ID NO: 4) or a fragment thereof which is capable of inhibiting growth of or killing Campylobacter spp;
e) a peptide comprising the amino acid sequence KAIAFQSSPGSPVLL (SEQ ID NO: 5) or a fragment thereof which is capable of inhibiting growth of or killing Campylobacter spp;
f) a peptide comprising the amino acid sequence TCNLSDYTLPRARVL (SEQ ID NO: 6) or a fragment thereof which is capable of inhibiting growth of or killing Campylobacter spp;
g) a peptide comprising the amino acid sequence NFLCPSPYPKVQPGL (SEQ ID NO: 7) or a fragment thereof which is capable of inhibiting growth of or killing Campylobacter spp;
h) a peptide comprising the amino acid sequence VPESIHKASLVCYRF (SEQ ID NO: 8) or a fragment thereof which is capable of inhibiting growth of or killing Campylobacter spp;
i) a peptide comprising the amino acid sequence SRPIARFYTWSQNTT (SEQ ID NO: 9) or a fragment thereof which is capable of inhibiting growth of or killing Campylobacter spp;
j) a peptide comprising the amino acid sequence FSPFRISELVYTLHP (SEQ ID NO: 10) or a fragment thereof which is capable of inhibiting growth of or killing Campylobacter spp;
k) a peptide comprising the amino acid sequence LPFNLAKPELYIFVQ (SEQ ID NO: 11) or a fragment thereof which is capable of inhibiting growth of or killing Campylobacter spp;
l) a peptide comprising the amino acid sequence GRFLIRVTSSPLGPD (SEQ ID NO: 12) or a fragment thereof which is capable of inhibiting growth of or killing Campylobacter spp;
m) a peptide comprising the amino acid sequence TGSGLYLHQMVYLYQ (SEQ ID NO: 13) or a fragment thereof which is capable of inhibiting growth of or killing Campylobacter spp;
n) a peptide comprising the amino acid sequence FLIDSPLASIGPTSM (SEQ ID NO: 14) or a fragment thereof which is capable of inhibiting growth of or killing Campylobacter spp;
o) a peptide comprising the amino acid sequence FMIDSPLASIGPTSM (SEQ ID NO: 15) or a fragment thereof which is capable of inhibiting growth of or killing Campylobacter spp;
p) a peptide comprising the amino acid sequence FLSDPPAPPTSPGVV (SEQ ID NO: 16) or a fragment thereof which is capable of inhibiting growth of or killing Campylobacter spp;
q) a peptide comprising the amino acid sequence VLAWFSPLTLESSRL (SEQ ID NO: 17) or a fragment thereof which is capable of inhibiting growth of or killing Campylobacter spp;
r) a peptide comprising the amino acid sequence VIDLSGTRKSSSGTM (SEQ ID NO: 18) or a fragment thereof which is capable of inhibiting growth of or killing Campylobacter spp;
s) a peptide comprising the amino acid sequence VRKTTSHPPSYALLH (SEQ ID NO: 19) or a fragment thereof which is capable of inhibiting growth of or killing Campylobacter spp;
t) a peptide comprising the amino acid sequence TPPYRAALATPVLLL (SEQ ID NO: 20) or a fragment thereof which is capable of inhibiting growth of or killing Campylobacter spp;
u) a peptide comprising the amino acid sequence LSAPSPMFLPPVNPH (SEQ ID NO: 21) or a fragment thereof which is capable of inhibiting growth of or killing Campylobacter spp;
v) a peptide comprising the amino acid sequence HRPVKTPANAPTTMM (SEQ ID NO: 22) or a fragment thereof which is capable of inhibiting growth of or killing Campylobacter spp;
w) a peptide comprising the amino acid sequence CFNDPLDIVPPMLLL (SEQ ID NO: 23) or a fragment thereof which is capable of inhibiting growth of or killing Campylobacter spp;
x) a peptide comprising the amino acid sequence AHAPCSLFFPLSLRP (SEQ ID NO: 24) or a fragment thereof which is capable of inhibiting growth of or killing Campylobacter spp;
y) a peptide comprising the amino acid sequence KALYALHVPSMQVFA (SEQ ID NO: 25) or a fragment thereof which is capable of inhibiting growth of or killing Campylobacter spp;
z) a peptide comprising the amino acid sequence RPSRWPWQEPLPISI (SEQ ID NO: 26) or a fragment thereof which is capable of inhibiting growth of or killing Campylobacter spp; and
aa) a peptide comprising the amino acid sequence KVQIIPKDTLAPLPP (SEQ ID NO: 27) or a fragment thereof which is capable of inhibiting growth of or killing Campylobacter spp.

4. A method according to claim 3 comprising contacting the Campylobacter spp. with a peptide comprising the amino acid sequence GRFLIRVTSSPLGPD (SEQ ID NO: 12) or a fragment thereof which is capable of inhibiting growth of or killing Campylobacter spp.

5. A method according to claim 3 comprising contacting the Campylobacter spp. with a peptide comprising the amino acid sequence TGSGLYLHQMVYLYQ (SEQ ID NO: 13) or a fragment thereof which is capable of inhibiting growth of or killing Campylobacter spp.

6. A method according to claim 3 comprising contacting the Campylobacter spp. with a peptide comprising the amino acid sequence FLIDSPLASIGPTSM (SEQ ID NO: 14) or a fragment thereof which is capable of inhibiting growth of or killing Campylobacter spp.

7. A method according to claim 3 comprising contacting the Campylobacter spp, with a peptide comprising the amino acid sequence FSPFRISELVYTLHP (SEQ ID NO: 10) or a fragment thereof which is capable of inhibiting growth of or killing Campylobacter spp.

8. A method according to claim 3 comprising contacting the Campylobacter spp. with a peptide comprising the amino acid sequence FLSDPPAPPTSPGVV (SEQ ID NO: 16) or a fragment thereof which is capable of inhibiting growth of or killing Campylobacter spp.

9. A method according to any one of claims 3 to 8, wherein the one or more peptides bind to the Campylobacter species to inhibit growth of or kill the Campylobacter species.

10. A method according to claim 9, wherein the one or more peptides bind specifically to the Campylobacter species to inhibit growth of or kill the Campylobacter species.

11. A method according to any one of claims 3 to 10, wherein the Campylobacter spp, is Campylobacter jejuni.

12. A method according to any one of claims 3 to 10, wherein the Campylobacter spp. is Campylobacter coll.

13. A method according to any one of claims 3 to 12, wherein the method comprises inhibiting growth of or killing Campylobacter spp. in a solution.

14. A method according to any one of claims 3 to 12, wherein the method comprises inhibiting growth of or killing Campylobacter spp. on a surface.

15. A method of preventing a Campylobacter spp. infection in a subject comprising administering to the subject one or more peptides in an amount sufficient to prevent infection in the subject, wherein the one or more peptides are selected from the group consisting of:
a) a peptide comprising the amino acid sequence EGVLGAALSAFSFDS (SEQ ID NO: 1) or a fragment thereof which is capable of preventing Campylobacter spp infection;
b) a peptide comprising the amino acid sequence QAQPCPNQPDGSVYA (SEQ ID NO: 2) or a fragment thereof which is capable of preventing Campylobacter spp infection;
c) a peptide comprising the amino acid sequence STALPLWSNYSYDSA (SEQ ID NO: 3) or a fragment thereof which is capable of preventing Campylobacter .spp infection;
d) a peptide comprising the amino acid sequence RTFGAKPPNIPFPRR (SEQ ID NO: 4) or a fragment thereof which is capable of preventing Campylobacter spp infection;
e) a peptide comprising the amino acid sequence KAIAFQSSPGSPVLL (SEQ ID NO: 5) or a fragment thereof which is capable of preventing Campylobacter spp infection;
f) a peptide comprising the amino acid sequence TCNLSDYTLPRARVL (SEQ ID NO: 6) or a fragment thereof which is capable of preventing Campylobacter spp infection;
g) a peptide comprising the amino acid sequence NFLCPSPYPKVQPGL (SEQ ID NO: 7) or a fragment thereof which is capable of preventing Campylobacter spp infection;
h) a peptide comprising the amino acid sequence VPESIHKASLVCYRF (SEQ ID NO: 8) or a fragment thereof which is capable of preventing Campylobacter spp infection;
i) a peptide comprising the amino acid sequence SRPIARFYTWSQNTT (SEQ ID NO: 9) or a fragment thereof which is capable of preventing Campylobacter spp infection;
j) a peptide comprising the amino acid sequence FSPFRISELVYTLHP (SEQ ID NO: 10) or a fragment thereof which is capable preventing Campylobacter spp infection;
k) a peptide comprising the amino acid sequence LPFNLAKPELYIFVQ (SEQ ID NO: 11) or a fragment thereof which is capable of preventing Campylobacter spp infection;
l) a peptide comprising the amino acid sequence GRFLIRVTSSPLGPD (SEQ ID NO: 12) or a fragment thereof which is capable of preventing Campylobacter spp infection;
m) a peptide comprising the amino acid sequence TGSGLYLHQMVYLYQ (SEQ ID NO: 13) or a fragment thereof which is capable of preventing Campylobacter spp infection;
n) a peptide comprising the amino acid sequence FLIDSPLASIGPTSM (SEQ ID NO: 14) or a fragment thereof which is capable of preventing Campylobacter spp infection;
o) a peptide comprising the amino acid sequence FMIDSPLASIGPTSM (SEQ ID NO: 15) or a fragment thereof which is capable of preventing Campylobacter spp infection;
p) a peptide comprising the amino acid sequence FLSDPPAPPTSPGVV (SEQ ID NO: 16) or a fragment thereof which is capable of preventing Campylobacter spp infection;
q) a peptide comprising the amino acid sequence VLAWFSPLTLESSRL (SEQ ID NO: 17) or a fragment thereof which is capable of preventing Campylobacter spp infection;
r) a peptide comprising the amino acid sequence VIDLSGTRKSSSGTM (SEQ ID NO: 18) or a fragment thereof which is capable of preventing Campylobacter spp infection;
s) a peptide comprising the amino acid sequence VRKTTSHPPSYALLH (SEQ ID NO: 19) or a fragment thereof which is capable of preventing Campylobacter spp infection;
t) a peptide comprising the amino acid sequence TPPYRAALATPVLLL (SEQ ID NO: 20) or a fragment thereof which is capable of preventing Campylobacter spp infection;
u) a peptide comprising the amino acid sequence LSAPSPMFLPPVNPH (SEQ ID NO: 21) or a fragment thereof which is capable of preventing Campylobacter spp infection;
v) a peptide comprising the amino acid sequence HRPVKTPANAPTTMM (SEQ ID NO: 22) or a fragment thereof which is capable of preventing Campylobacter spp infection;
w) a peptide comprising the amino acid sequence CFNDPLDIVPPMLLL (SEQ ID NO: 23) or a fragment thereof which is capable of preventing Campylobacter spp infection;
x) a peptide comprising the amino acid sequence AHAPCSLFFPLSLRP (SEQ ID NO: 24) or a fragment thereof which is capable of preventing Campylobacter spp infection;
y) a peptide comprising the amino acid sequence KALYALHVPSMQVFA (SEQ ID NO: 25) or a fragment thereof which is capable of preventing Campylobacter spp infection;
z) a peptide comprising the amino acid sequence RPSRWPWQEPLPISI (SEQ ID NO: 26) or a fragment thereof which is capable of preventing Campylobacter spp infection; and
aa) a peptide comprising the amino acid sequence KVQIIPKDTLAPLPP (SEQ ID NO: 27) or a fragment thereof which is capable of preventing Campylobacter spp infection.

16. A method according to claim 15 comprising contacting the Campylobacter spp. with a peptide comprising the amino acid sequence GRFLIRVTSSPLGPD (SEQ ID NO: 12) or a fragment thereof which is capable of preventing Campylobacter spp. infection.

17. A method according to claim 15 comprising contacting the Campylobacter spp. with a peptide comprising the amino acid sequence TGSGLYLHQMVYLYQ (SEQ ID NO: 13) or a fragment thereof which is capable of preventing Campylobacter spp. infection.

18. A method according to claim 15 comprising contacting the Campylobacter spp. with a peptide comprising the amino acid sequence FLIDSPLASIGPTSM (SEQ ID NO: 14) or a fragment thereof which is capable of preventing Campylobacter spp. infection.

19. A method according to claim 15 comprising contacting the Campylobacter spp. with a peptide comprising the amino acid sequence FSPFRISELVYTLHP (SEQ ID NO: 10) or a fragment thereof which is capable of preventing Campylobacter spp. infection.

20. A method according to claim 15 comprising contacting the Campylobacter spp. with a peptide comprising the amino acid sequence FLSDPPAPPTSPGVV (SEQ ID NO: 16) or a fragment thereof which is capable of preventing Campylobacter spp. infection.

21. A method of treating a Campylobacter spp. infection in a subject comprising administering to the subject one or more peptides in an amount sufficient to treat the infection in the subject, wherein the one or more peptides are selected from the group consisting of:
a) a peptide comprising the amino acid sequence EGVLGAALSAFSFDS (SEQ ID NO: 1) or a fragment thereof which is capable of treating Campylobacter spp infection;
b) a peptide comprising the amino acid sequence QAQPCPNQPDGSVYA (SEQ ID NO: 2) or a fragment thereof which is capable of treating Campylobacter spp infection; c) a peptide comprising the amino acid sequence STALPLWSNYSYDSA (SEQ ID NO: 3) or a fragment thereof which is capable of treating Campylobacter spp infection;
d) a peptide comprising the amino acid sequence RTFGAKPPNIPFPR1Z. (SEQ ID NO: 4) or a fragment thereof which is capable of treating Campylobacter spp infection;
e) a peptide comprising the amino acid sequence KAIAFQSSPGSPVLL (SEQ ID NO: 5) or a fragment thereof which is capable of treating Campylobacter spp infection;
f) a peptide comprising the amino acid sequence TCNLSDYTLPRARVL (SEQ ID NO: 6) or a fragment thereof which is capable of treating Campylobacter spp infection;
g) a peptide comprising the amino acid sequence NFLCPSPYPKVQPGL (SEQ ID NO: 7) or a fragment thereof which is capable of treating Campylobacter spp infection;
h) a peptide comprising the amino acid sequence VPESIHKASLVCYRF (SEQ ID NO: 8) or a fragment thereof which is capable of treating Campylobacter spp infection;
i) a peptide comprising the amino acid sequence SRPIARFYTWSQNTT (SEQ ID NO: 9) or a fragment thereof which is capable of treating Campylobacter spp infection;
j) a peptide comprising the amino acid sequence FSPFRISELVYTLHP (SEQ ID NO: 10) or a fragment thereof which is capable treating Campylobacter spp infection;
k) a peptide comprising the amino acid sequence LPFNLAKPELYIFVQ (SEQ ID NO: 11) or a fragment thereof which is capable of treating Campylobacter spp infection;
l) a peptide comprising the amino acid sequence GRFLIRVTSSPLGPD (SEQ ID NO: 12) or a fragment thereof which is capable of treating Campylobacter spp infection;
m) a peptide comprising the amino acid sequence TGSGLYLHQMVYLYQ (SEQ ID NO: 13) or a fragment thereof which is capable of treating Campylobacter spp infection;
n) a peptide comprising the amino acid sequence FLIDSPLASIGPTSM (SEQ ID NO: 14) or a fragment thereof which is capable of treating Campylobacter spp infection;
o) a peptide comprising the amino acid sequence FMIDSPLASIGPTSM (SEQ ID NO: 15) or a fragment thereof which is capable of treating Campylobacter spp infection;
p) a peptide comprising the amino acid sequence FLSDPPAPPTSPGVV (SEQ ID NO: 16) or a fragment thereof which is capable of treating Campylobacter spp infection;
q) a peptide comprising the amino acid sequence VLAWFSPLTLESSRL (SEQ ID NO: 17) or a fragment thereof which is capable of treating Campylobacter spp infection;
r) a peptide comprising the amino acid sequence VIDLSGTRKSSSGTM (SEQ ID NO: 18) or a fragment thereof which is capable of treating Campylobacter spp infection;
s) a peptide comprising the amino acid sequence VRKTTSHPPSYALLH (SEQ ID NO: 19) or a fragment thereof which is capable of treating Campylobacter spp infection;
t) a peptide comprising the amino acid sequence TPPYRAALATPVLLL (SEQ ID NO: 20) or a fragment thereof which is capable of treating Campylobacter spp infection;
u) a peptide comprising the amino acid sequence LSAPSPMFLPPVNPH (SEQ ID NO: 21) or a fragment thereof which is capable of treating Campylobacter spp infection;
v) a peptide comprising the amino acid sequence HRPVKTPANAPTTMM (SEQ ID NO: 22) or a fragment thereof which is capable of treating Campylobacter spp infection;
w) a peptide comprising the amino acid sequence CFNDPLDIVPPMLLL (SEQ ID NO: 23) or a fragment thereof which is capable of treating Campylobacter spp infection;
x) a peptide comprising the amino acid sequence AHAPCSLFFPLSLRP (SEQ ID NO: 24) or a fragment thereof which is capable of treating Campylobacter spp infection;
y) a peptide comprising the amino acid sequence KALYALHVPSMQVFA (SEQ ID NO: 25) or a fragment thereof which is capable of treating Campylobacter spp infection;
z) a peptide comprising the amino acid sequence RPSRWPWQEPLPISI (SEQ ID NO: 26) or a fragment thereof which is capable of treating Campylobacter spp infection; and
aa) a peptide comprising the amino acid sequence KVQIIPKDTLAPLPP (SEQ ID NO: 27) or a fragment thereof which is capable of treating Campylobacter spp infection.

22. A method according to claim 21 comprising contacting the Campylobacter spp. with a peptide comprising the amino acid sequence GRFLIRVTSSPLGPD (SEQ ID NO: 12) or a fragment thereof which is capable of treating Campylobacter app. infection.

23. A method according to claim 21 comprising contacting the Campylobacter spp. with a peptide comprising the amino acid sequence TGSGLYLHQMVYLYQ (SEQ ID NO: 13) or a fragment thereof which is capable of treating Campylobacter spp. infection.

24. A method according to claim 21 comprising contacting the Campylobacter spp. with a peptide comprising the amino acid sequence FLIDSPLASIGPTSM (SEQ ID NO: 14) or a fragment thereof which is capable of treating Campylobacter app. infection.

25. A method according to claim 21 comprising contacting the Campylobacter spp. with a peptide comprising the amino acid sequence FSPFRISELVYTLHP (SEQ ID NO: 10) or a fragment thereof which is capable of treating Campylobacter spp. infection.

26. A method according to claim 21 comprising contacting the Campylobacter spp. with a peptide comprising the amino acid sequence FLSDPPAPPTSPGVV (SEQ ID NO: 16) or a fragment thereof which is capable of treating Campylobacter spp. infection.

27. A method of diagnosing a Campylobacter spp. infection in a subject comprising:
1) providing a sample from the subject;
2) contacting the sample with one or more peptides under conditions which permit binding of the one or more peptides to Campylobacter sp.; and
3) determining if the one or more peptides bind to a Campylobacter spp. in the sample, wherein the one or more peptides are selected from the group consisting of:
a) a peptide comprising the amino acid sequence EGVLGAALSAFSFDS (SEQ ID NO: 1) or a fragment thereof which is capable of diagnosing Campylobacter spp infection; b) a peptide comprising the amino acid sequence QAQPCPNQPDGSVYA (SEQ ID NO: 2) or a fragment thereof which is capable of diagnosing Campylobacter spp infection;
c) a peptide comprising the amino acid sequence STALPLWSNYSYDSA (SEQ ID NO: 3) or a fragment thereof which is capable of diagnosing Campylobacter spp infection; d) a peptide comprising the amino acid sequence RTFGAKPPNIPFPRR (SEQ ID NO: 4) or a fragment thereof which is capable of diagnosing Campylobacter spp infection;
e) a peptide comprising the amino acid sequence KAIAFQSSPGSPVLL (SEQ ID NO: 5) or a fragment thereof which is capable of diagnosing Campylobacter spp infection;
f) a peptide comprising the amino acid sequence TCNLSDYTLPRARVL (SEQ ID NO: 6) or a fragment thereof which is capable of diagnosing Campylobacter spp infection; g) a peptide comprising the amino acid sequence NFLCPSPYPKVQPGL (SEQ ID NO: 7) or a fragment thereof which is capable of diagnosing Campylobacter spp infection; h) a peptide comprising the amino acid sequence VPESIHKASLVCYRF (SEQ ID NO: 8) or a fragment thereof which is capable of diagnosing Campylobacter spp infection;
i) a peptide comprising the amino acid sequence SRPIARFYTWSQNTT (SEQ ID NO: 9) or a fragment thereof which is capable of diagnosing Campylobacter spp infection;
j) a peptide comprising the amino acid sequence FSPFRISELVYTLHP (SEQ ID NO: 10) or a fragment thereof which is capable diagnosing Campylobacter spp infection;
k) a peptide comprising the amino acid sequence LPFNLAKPELYIFVQ (SEQ ID NO: 11) or a fragment thereof which is capable of diagnosing Campylobacter spp infection;
l) a peptide comprising the amino acid sequence GRFLIRVTSSPLGPD (SEQ ID NO: 12) or a fragment thereof which is capable of diagnosing Campylobacter spp infection;
m) a peptide comprising the amino acid sequence TGSGLYLHQMVYLYQ (SEQ ID NO: 13) or a fragment thereof which is capable of diagnosing Campylobacter spp infection;
n) a peptide comprising the amino acid sequence FLIDSPLASIGPTSM (SEQ ID NO: 14) or a fragment thereof which is capable of diagnosing Campylobacter spp infection;
o) a peptide comprising the amino acid sequence FMIDSPLASIGPTSM (SEQ ID NO: 15) or a fragment thereof which is capable of diagnosing Campylobacter spp infection;
p) a peptide comprising the amino acid sequence FLSDPPAPPTSPGVV (SEQ ID NO: 16) or a fragment thereof which is capable of diagnosing Campylobacter spp infection;
q) a peptide comprising the amino acid sequence VLAWFSPLTLESSRL (SEQ ID NO: 17) or a fragment thereof which is capable of diagnosing Campylobacter spp infection;
r) a peptide comprising the amino acid sequence VIDLSGTRKSSSGTM (SEQ ID NO: 18) or a fragment thereof which is capable of diagnosing Campylobacter spp infection;
s) a peptide comprising the amino acid sequence VRKTTSHPPSYALLH (SEQ ID NO: 19) or a fragment thereof which is capable of diagnosing Campylobacter spp infection;
t) a peptide comprising the amino acid sequence TPPYRAALATPVLLL (SEQ ID NO: 20) or a fragment thereof which is capable of diagnosing Campylobacter spp infection;
u) a peptide comprising the amino acid sequence LSAPSPMFLPPVNPH (SEQ ID NO: 21) or a fragment thereof which is capable of diagnosing Campylobacter spp infection;
v) a peptide comprising the amino acid sequence HRPVKTPANAPTTMM (SEQ ID NO: 22) or a fragment thereof which is capable of diagnosing Campylobacter spp infection;
w) a peptide comprising the amino acid sequence CFNDPLDIVPPMLLL (SEQ ID NO: 23) or a fragment thereof which is capable of diagnosing Campylobacter spp infection;
x) a peptide comprising the amino acid sequence AHAPCSLFFPLSLRP (SEQ ID NO: 24) or a fragment thereof which is capable of diagnosing Campylobacter spp infection;
y) a peptide comprising the amino acid sequence KALYALHVPSMQVFA (SEQ ID NO: 25) or a fragment thereof which is capable of diagnosing Campylobacter spp infection;
z) a peptide comprising the amino acid sequence RPSRWPWQEPLPISI (SEQ ID NO: 26) or a fragment thereof which is capable of diagnosing Campylobacter spp infection; and
aa) a peptide comprising the amino acid sequence KVQIIPKDTLAPLPP (SEQ ID NO: 27) or a fragment thereof which is capable of diagnosing Campylobacter spp infection.

28. A method according to claim 27 comprising contacting the sample with a peptide comprising the amino acid sequence FMIDSPLASIGPTSM (SEQ ID NO: 15) or a fragment thereof which is capable of diagnosing Campylobacter spp infection.

29. A method according to claim 28 comprising contacting the sample with a peptide comprising the amino acid sequence FLSDPPAPPTSPGVV (SEQ ID NO: 16) or a fragment thereof which is capable of diagnosing Campylobacter spp infection.

30. A method according to any one of claims 15 to 29, wherein the Campylobacter spp. is Campylobacter jejuni.

31. A method according to any one of claims 15 to 29, wherein the Campylobacter spp. is Campylobacter

32. A method according to any one of claims 15 to 31, wherein the subject in selected from the group consisting of humans, poultry, domesticated animals or agricultural animals.

33. A method according to claim 32, wherein poultry comprises chickens, ducks, geese or turkeys.

34. A method according to claim 32, wherein domesticated animals comprise cats or dogs

35. A method according to claim 32, wherein agricultural animals comprise pigs, cattle or sheep.

36. An isolated peptide comprising an amino acid sequence selected from the group consisting of: EGVLGAALSAFSFDS (SEQ ID NO: 1); QAQPCPNQPDGSVYA (SEQ ID NO: 2); STALPLWSNYSYDSA (SEQ ID NO: 3); RTFGAKPPNIPFPRR (SEQ ID NO: 4); KAIAFQSSPGSPVLL (SEQ ID NO: 5); TCNLSDYTLPRARVL (SEQ ID NO: 6); NFLCPSPYPKVQPGL (SEQ ID NO: 7); VPESIHKASLVCYRF (SEQ ID NO: 8); SRPIARFYTWSQNTT (SEQ ID NO: 9); FSPFRISELVYTLHP (SEQ ID NO: 10); LPFNLAKPELYIFVQ (SEQ ID NO: 11); GRFLIRVTSSPLGPD (SEQ ID NO: 12); TGSGLYLHQMVYLYQ (SEQ ID NO: 13); FLIDSPLASIGPTSM (SEQ ID NO: 14) FMIDSPLASIGPTSM (SEQ ID NO: 15); FLSDPPAPPTSPGVV (SEQ ID NO: 16); VLAWFSPLTLESSRL (SEQ ID NO: 17); VIDLSGTRKSSSGTM (SEQ ID NO: 18); VRKTTSHPPSYALLH (SEQ ID NO: 19); TPPYRAALATPVLLL (SEQ ID NO: 20); LSAPSPMFLPPVNPH (SEQ ID NO: 21); HRPVKTPANAPTTMM (SEQ ID NO: 22); CFNDPLDIVPPMLLL (SEQ ID NO: 23); AHAPCSLFFPLSLRP (SEQ ID NO: 24); KALYALHVPSMQVFA (SEQ ID NO: 25); RPSRWPWQEPLPISI (SEQ ID NO: 26); and KVQIIPKDTLAPLPP (SEQ ID NO: 27).

37. An isolated peptide of 15 to 100 amino acid residues comprising an amino acid sequence selected from the group consisting of: EGVLGAALSAFSFDS (SEQ ID NO: 1); QAQPCPNQPDGSVYA (SEQ ID NO: 2); STALPLWSNYSYDSA (SEQ ID NO: 3); RTFGAKPPNIPFPRR (SEQ ID NO: 4); KAIAFQSSPGSPVLL (SEQ ID NO: 5); TCNLSDYTLPRARVL (SEQ ID NO: 6); NFLCPSPYPKVQPGL (SEQ ID NO: 7); VPESIHKASLVCYRF (SEQ ID NO: 8); SRPIARFYTWSQNTT (SEQ ID NO: 9); FSPFRISELVYTLHP (SEQ ID NO: 10); LPFNLAKPELYIFVQ (SEQ ID NO: 11); GRFLIRVTSSPLGPD (SEQ ID NO: 12); TGSGLYLHQMVYLYQ (SEQ ID NO: 13); FLIDSPLASIGPTSM (SEQ ID NO: 14) FMIDSPLASIGPTSM (SEQ ID NO: 15); FLSDPPAPPTSPGVV (SEQ ID NO: 16); VLAWFSPLTLESSRL (SEQ ID NO: 17); VIDLSGTRKSSSGTM (SEQ ID NO: 18); VRKTTSHPPSYALLH (SEQ ID NO: 19); TPPYRAALATPVLLL (SEQ ID NO: 20); LSAPSPMFLPPVNPH (SEQ ID NO: 21); HRPVKTPANAPTTMM (SEQ ID NO: 22); CFNDPLDIVPPMLLL (SEQ ID NO: 23); AHAPCSLFFPLSLRP (SEQ ID NO: 24); KALYALHVPSMQVFA (SEQ ID NO: 25); RPSRWPWQEPLPISI (SEQ ID NO: 26); and KVQIIPKDTLAPLPP (SEQ ID NO: 27).

38. A synthetic peptide comprising an amino acid sequence selected from the group consisting of: EGVLGAALSAFSFDS (SEQ ID NO: 1); QAQPCPNQPDGSVYA (SEQ ID NO: 2); STALPLWSNYSYDSA (SEQ ID NO: 3); RTFGAKPPNIPFPRR (SEQ ID NO: 4); KAIAFQSSPGSPVLL (SEQ ID NO: 5); TCNLSDYTLPRARVL (SEQ ID NO: 6); NFLCPSPYPKVQPGL (SEQ ID NO: 7); VPESIHKASLVCYRF (SEQ ID NO: 8); SRPIARFYTWSQNTT (SEQ ID NO: 9); FSPFRISELVYTLHP (SEQ ID NO: 10); LPFNLAKPELYIFVQ (SEQ ID NO: 11); GRFLIRVTSSPLGPD (SEQ ID NO: 12); TGSGLYLHQMVYLYQ (SEQ ID NO: 13); FLIDSPLASIGPTSM (SEQ ID NO: 14) FMIDSPLASIGPTSM (SEQ ID NO: 15); FLSDPPAPPTSPGVV (SEQ ID NO: 16); VLAWFSPLTLESSRL (SEQ ID NO: 17); VIDLSGTRKSSSGTM (SEQ ID NO: 18); VRKTTSHPPSYALLH (SEQ ID NO: 19); TPPYRAALATPVLLL (SEQ ID NO: 20); LSAPSPMFLPPVNPH (SEQ ID NO: 21); HRPVKTPANAPTTMM (SEQ ID NO: 22); CFNDPLDIVPPMLLL (SEQ ID NO: 23); AHAPCSLFFPLSLRP (SEQ ID NO: 24); KALYALHVPSMQVFA (SEQ ID NO: 25); RPSRWPWQEPLPISI (SEQ ID NO: 26); and KVQIIPKDTLAPLPP (SEQ ID NO: 27).

39. A composition comprising one or more peptides, wherein the one of more peptides comprise an amino acid sequence selected from the group consisting of:

| | |
|---|---|
| EGVLGAALSAFSFDS; | (SEQ ID NO: 1) |
| QAQPCPNQPDGSVYA; | (SEQ ID NO: 2) |
| STALPLWSNYSYDSA; | (SEQ ID NO: 3) |
| RTFGAKPPNIPFPRR; | (SEQ ID NO: 4) |
| KAIAFQSSPGSPVLL; | (SEQ ID NO: 5) |
| TCNLSDYTLPRARVL; | (SEQ ID NO: 6) |
| NFLCPSPYPKVQPGL; | (SEQ ID NO: 7) |
| VPESIHKASLVCYRF; | (SEQ ID NO: 8) |
| SRPIARFYTWSQNTT; | (SEQ ID NO: 9) |
| FSPFRISELVYTLHP; | (SEQ ID NO: 10) |
| LPFNLAKPELYIFVQ; | (SEQ ID NO: 11) |
| GRFLIRVTSSPLGPD; | (SEQ ID NO: 12) |
| TGSGLYLHQMVYLYQ; | (SEQ ID NO: 13) |
| FLIDSPLASIGPTSM | (SEQ ID NO: 14) |
| FMIDSPLASIGPTSM; | (SEQ ID NO: 15) |
| FLSDPPAPPTSPGVV; | (SEQ ID NO: 16) |
| VLAWFSPLTLESSRL; | (SEQ ID NO: 17) |
| VIDLSGTRKSSSGTM; | (SEQ ID NO: 18) |
| VRKTTSHPPSYALLH; | (SEQ ID NO: 19) |
| TPPYRAALATPVLLL; | (SEQ ID NO: 20) |
| LSAPSPMFLPPVNPH; | (SEQ ID NO: 21) |
| HRPVKTPANAPTTMM; | (SEQ ID NO: 22) |
| CFNDPLDIVPPMLLL; | (SEQ ID NO: 23) |
| AHAPCSLFFPLSLRP; | (SEQ ID NO: 24) |
| KALYALHVPSMQVFA; | (SEQ ID NO: 25) |
| RPSRWPWQEPLPISI; | (SEQ ID NO: 26) |
| and | |
| KVQIIPKDTLAPLPP. | (SEQ ID NO: 27) |

40. A pharmaceutical composition comprising one or more peptides and a pharmaceutically acceptable carrier, wherein the one or more peptides comprise an amino acid sequence selected from the group consisting of: EGVLGAALSAFSFDS (SEQ ID NO: 1); QAQPCPNQPDGSVYA (SEQ ID NO: 2); STALPLWSNYSYDSA (SEQ ID NO: 3); RTFGAKPPNIPFPRR (SEQ ID NO: 4); KAIAFQSSPGSPVLL (SEQ ID NO: 5); TCNLSDYTLPRARVL (SEQ ID NO: 6); NFLCPSPYPKVQPGL (SEQ ID NO: 7); VPESIHKASLVCYRF (SEQ ID NO: 8); SRPIARFYTWSQNTT (SEQ ID NO: 9); FSPFRISELVYTLHP (SEQ ID NO: 10); LPFNLAKPELYIFVQ (SEQ ID NO: 11); GRFLIRVTSSPLGPD (SEQ ID NO: 12); TGSGLYLHQMVYLYQ (SEQ ID NO: 13); FLIDSPLASIGPTSM (SEQ ID NO: 14) FMIDSPLASIGPTSM (SEQ ID NO: 15); FLSDPPAPPTSPGVV (SEQ ID NO: 16); VLAWFSPLTLESSRL (SEQ ID NO: 17); VIDLSGTRKSSSGTM (SEQ ID NO: 18); VRKTTSHPPSYALLH (SEQ ID NO: 19); TPPYRAALATPVLLL (SEQ ID NO: 20); LSAPSPMFLPPVNPH (SEQ ID NO: 21); HRPVKTPANAPTTMM (SEQ ID NO: 22); CFNDPLDIVPPMLLL (SEQ ID NO: 23); AHAPCSLFFPLSLRP (SEQ ID NO: 24); KALYALHVPSMQVFA (SEQ ID NO: 25); RPSRWPWQEPLPISI (SEQ ID NO: 26); and KVQIIPKDTLAPLPP (SEQ ID NO: 27).

41. A fusion protein comprising one or more peptides, wherein the one or more peptides comprise an amino acid sequence selected from the group consisting of:

| | |
|---|---|
| EGVLGAALSAFSFDS; | (SEQ ID NO: 1) |
| QAQPCPNQPDGSVYA; | (SEQ ID NO: 2) |
| STALPLWSNYSYDSA; | (SEQ ID NO: 3) |
| RTFGAKPPNIPFPRR; | (SEQ ID NO: 4) |
| KAIAFQSSPGSPVLL; | (SEQ ID NO: 5) |
| TCNLSDYTLPRARVL; | (SEQ ID NO: 6) |
| NFLCPSPYPKVQPGL; | (SEQ ID NO: 7) |
| VPESIHKASLVCYRF; | (SEQ ID NO: 8) |
| SRPIARFYTWSQNTT; | (SEQ ID NO: 9) |
| FSPFRISELVYTLHP; | (SEQ ID NO: 10) |
| LPFNLAKPELYIFVQ; | (SEQ ID NO: 11) |
| GRFLIRVTSSPLGPD; | (SEQ ID NO: 12) |
| TGSGLYLHQMVYLYQ; | (SEQ ID NO: 13) |
| FLIDSPLASIGPTSM | (SEQ ID NO: 14) |
| FMIDSPLASIGPTSM; | (SEQ ID NO: 15) |
| FLSDPPAPPTSPGVV; | (SEQ ID NO: 16) |
| VLAWFSPLTLESSRL; | (SEQ ID NO: 17) |
| VIDLSGTRKSSSGTM; | (SEQ ID NO: 18) |
| VRKTTSHPPSYALLH; | (SEQ ID NO: 19) |
| TPPYRAALATPVLLL; | (SEQ ID NO: 20) |
| LSAPSPMFLPPVNPH; | (SEQ ID NO: 21) |
| HRPVKTPANAPTTMM; | (SEQ ID NO: 22) |
| CFNDPLDIVPPMLLL; | (SEQ ID NO: 23) |
| AHAPCSLFFPLSLRP; | (SEQ ID NO: 24) |
| KALYALHVPSMQVFA; | (SEQ ID NO: 25) |
| RPSRWPWQEPLPISI; | (SEQ ID NO: 26) |
| and | |
| KVQIIPKDTLAPLPP. | (SEQ ID NO: 27) |

42. A fusion protein according to claim 41, wherein the protein further comprises a phage coat or part thereof.

43. A fd-tet phage comprising one or more peptides, wherein the one or more peptides comprise an amino acid sequence selected from the group consisting of:

| | |
|---|---|
| EGVLGAALSAFSFDS; | (SEQ ID NO: 1) |
| QAQPCPNQPDGSVYA; | (SEQ ID NO: 2) |
| STALPLWSNYSYDSA; | (SEQ ID NO: 3) |
| RTFGAKPPNIPFPRR; | (SEQ ID NO: 4) |
| KAIAFQSSPGSPVLL; | (SEQ ID NO: 5) |
| TCNLSDYTLPRARVL; | (SEQ ID NO: 6) |
| NFLCPSPYPKVQPGL; | (SEQ ID NO: 7) |
| VPESIHKASLVCYRF; | (SEQ ID NO: 8) |
| SRPIARFYTWSQNTT; | (SEQ ID NO: 9) |
| FSPFRISELVYTLHP; | (SEQ ID NO: 10) |
| LPFNLAKPELYIFVQ; | (SEQ ID NO: 11) |
| GRFLIRVTSSPLGPD; | (SEQ ID NO: 12) |
| TGSGLYLHQMVYLYQ; | (SEQ ID NO: 13) |
| FLIDSPLASIGPTSM, | (SEQ ID NO: 14) |
| FMIDSPLASIGPTSM; | (SEQ ID NO: 15) |
| FLSDPPAPPTSPGVV; | (SEQ ID NO: 16) |
| VLAWFSPLTLESSRL; | (SEQ ID NO: 17) |
| VIDLSGTRKSSSGTM; | (SEQ ID NO: 18) |
| VRKTTSHPPSYALLH; | (SEQ ID NO: 19) |
| TPPYRAALATPVLLL; | (SEQ ID NO: 20) |
| LSAPSPMFLPPVNPH; | (SEQ ID NO: 21) |
| HRPVKTPANAPTTMM; | (SEQ ID NO: 22) |
| CFNDPLDIVPPMLLL; | (SEQ ID NO: 23) |
| AHAPCSLFFPLSLRP; | (SEQ ID NO: 24) |
| KALYALHVPSMQVFA; | (SEQ ID NO: 25) |
| RPSRWPWQEPLPISI; | (SEQ ID NO: 26) |
| and | |
| KVQIIPKDTLAPLPP. | (SEQ ID NO: 27) |

44. An isolated nucleic acid molecule which encodes a peptide selected from the group consisting of: EGVLGAALSAFSFDS (SEQ ID NO: 1); QAQPCPNQPDGSVYA (SEQ ID NO: 2); STALPLWSNYSYDSA (SEQ ID NO: 3); RTFGAKPPNIPFPRR (SEQ ID NO: 4); KAIAFQSSPGSPVLL (SEQ ID NO: 5); TCNLSDYTLPRARVL (SEQ ID NO: 6); NFLCPSPYPKVQPGL (SEQ ID NO: 7); VPESIHKASLVCYRF (SEQ ID NO: 8); SRPIARFYTWSQNTT (SEQ ID NO: 9); FSPFRISELVYTLHP (SEQ ID NO: 10); LPFNLAKPELYIFVQ (SEQ ID NO: 11); GRFLIRVTSSPLGPD (SEQ ID NO: 12); TGSGLYLHQMVYLYQ (SEQ ID NO: 13); FLIDSPLASIGPTSM (SEQ ID NO: 14), FMIDSPLASIGPTSM (SEQ ID NO: 15); FLSDPPAPPTSPGVV (SEQ ID NO: 16); VLAWFSPLTLESSRL (SEQ ID NO: 17); VIDLSGTRKSSSGTM (SEQ ID NO: 18); VRKTTSHPPSYALLH (SEQ ID NO: 19); TPPYRAALATPVLLL (SEQ ID NO: 20); LSAPSPMFLPPVNPH (SEQ ID NO: 21); HRPVKTPANAPTTMM (SEQ ID NO: 22); CFNDPLDIVPPMLLL (SEQ ID NO: 23); AHAPCSLFFPLSLRP (SEQ ID NO: 24); KALYALHVPSMQVFA (SEQ ID NO: 25); RPSRWPWQEPLPISI (SEQ ID NO: 26); and KVQIIPKDTLAPLPP (SEQ ID NO: 27).

45. The use of one or more peptides selected from the group consisting of:
a) a peptide comprising the amino acid sequence EGVLGAALSAFSFDS (SEQ ID NO: 1) or a fragment thereof which is capable of treating Campylobacter spp infection;
b) a peptide comprising the amino acid sequence QAQPCPNQPDGSVYA (SEQ ID NO: 2) or a fragment thereof which is capable of treating Campylobacter spp infection;
c) a peptide comprising the amino acid sequence STALPLWSNYSYDSA (SEQ ID NO: 3) or a fragment thereof which is capable of treating Campylobacter spp infection;
d) a peptide comprising the amino acid sequence RTFGAKPPNIPFPRR (SEQ ID NO: 4) or a fragment thereof which is capable of treating Campylobacter .spp infection;
e) a peptide comprising the amino acid sequence KAIAFQSSPGSPVLL (SEQ ID NO: 5) or a fragment thereof which is capable of treating Campylobacter spp infection;
f) a peptide comprising the amino acid sequence TCNLSDYTLPRARVL (SEQ ID NO: 6) or a fragment thereof which is capable of treating Campylobacter spp infection;
g) a peptide comprising the amino acid sequence NFLCPSPYPKVQPGL (SEQ ID NO: 7) or a fragment thereof which is capable of treating Campylobacter spp infection;
h) a peptide comprising the amino acid sequence VPESIHKASLVCYRF (SEQ ID NO: 8) or a fragment thereof which is capable of treating Campylobacter spp infection;
i) a peptide comprising the amino acid sequence SRPIARFYTWSQNTT (SEQ ID NO: 9) or a fragment thereof which is capable of treating Campylobacter spp infection;
j) a peptide comprising the amino acid sequence FSPFRISELVYTLHP (SEQ ID NO: 10) or a fragment thereof which is capable treating Campylobacter spp infection;
k) a peptide comprising the amino acid sequence LPFNLAKPELYIFVQ (SEQ ID NO: 11) or a fragment thereof which is capable of treating Campylobacter spp infection;
l) a peptide comprising the amino acid sequence GRFLIRVTSSPLGPD (SEQ ID NO: 12) or a fragment thereof which is capable of treating Campylobacter spp infection;
m) a peptide comprising the amino acid sequence TGSGLYLHQMVYLYQ (SEQ ID NO: 13) or a fragment thereof which is capable of treating Campylobacter spp infection;
n) a peptide comprising the amino acid sequence FLIDSPLASIGPTSM (SEQ ID NO: 14) or a fragment thereof which is capable of treating Campylobacter spp infection;
o) a peptide comprising the amino acid sequence FMIDSPLASIGPTSM (SEQ ID NO: 15) or a fragment thereof which is capable of treating Campylobacter spp infection;
p) a peptide comprising the amino acid sequence FLSDPPAPPTSPGVV (SEQ ID NO: 16) or a fragment thereof which is capable of treating Campylobacter spp infection;
q) a peptide comprising the amino acid sequence VLAWFSPLTLESSRL (SEQ ID NO: 17) or a fragment thereof which is capable of treating Campylobacter spp infection;
r) a peptide comprising the amino acid sequence VIDLSGTRKSSSGTM (SEQ ID NO: 18) or a fragment thereof which is capable of treating Campylobacter spp infection;
s) a peptide comprising the amino acid sequence VRKTTSHPPSYALLH (SEQ ID NO: 19) or a fragment thereof which is capable of treating Campylobacter spp infection;
t) a peptide comprising the amino acid sequence TPPYRAALATPVLLL (SEQ ID NO: 20) or a fragment thereof which is capable of treating Campylobacter spp infection;
u) a peptide comprising the amino acid sequence LSAPSPMFLPPVNPH (SEQ ID NO: 21) or a fragment thereof which is capable of treating Campylobacter spp infection;
v) a peptide comprising the amino acid sequence HRPVKTPANAPTTMM (SEQ ID NO: 22) or a fragment thereof which is capable of treating Campylobacter spp infection;
w) a peptide comprising the amino acid sequence CFNDPLDIVPPMLLL (SEQ ID NO: 23) or a fragment thereof which is capable of treating Campylobacter spp infection;
x) a peptide comprising the amino acid sequence AHAPCSLFFPLSLRP (SEQ ID NO: 24) or a fragment thereof which is capable of treating Campylobacter spp infection;
y) a peptide comprising the amino acid sequence KALYALHVPSMQVFA (SEQ ID NO: 25) or a fragment thereof which is capable of treating Campylobacter spp infection;
z) a peptide comprising the amino acid sequence RPSRWPWQEPLPISI (SEQ ID NO: 26) or a fragment thereof which is capable of treating Campylobacter spp infection; and
aa) a peptide comprising the amino acid sequence KVQIIPKDTLAPLPP (SEQ ID NO: 27) or a fragment thereof which is capable of treating Campylobacter spp infection in the manufacture of a medicament for the treatment of Campylobacter spp infection in a subject.

46. Use according to claim 45 wherein the peptide comprises the amino acid sequence GRFLIRVTSSPLGPD (SEQ ID NO: 12) or a fragment thereof which is capable of treating Campylobacter spp infection.

47. Use according to claim 45 wherein the peptide comprises the amino acid sequence TGSGLYLHQMVYLYQ (SEQ ID NO: 13) or a fragment thereof which is capable of treating Campylobacter spp infection.

48. Use according to claim 45 wherein the peptide comprises the amino acid sequence FLIDSPLASIGPTSM (SEQ ID NO: 14) or a fragment thereof which is capable of treating Campylobacter spp infection.

49. Use according to claim 45 wherein the peptide comprises the amino acid sequence FSPFRISELVYTLHP (SEQ ID NO: 10) or a fragment thereof which is capable treating Campylobacter spp infection.

50. Use according to claim 45 wherein the peptide comprises the amino acid sequence FLSDPPAPPTSPGVV (SEQ ID NO: 16) or a fragment thereof which is capable of treating Campylobacter spp infection.
